SEQUENCE LISTING (45 sequences)

SEQ ID NO: 1 - DNA, 1467 bp, Homo sapiens
  CDS (5)..(793)
caccatgctcgccctggaggctgcacagctcgacgggccacacttcagctgtctgtacccagatggcgtcttctatgacctggacagctgcaagcattccagctaccctgattcagagggggctcctgactccctgtgggactggactgtggccccacctgtcccagccaccccctatgaagccttcgacccggcagcagccgcttttagccacccccaggctgcccagctctgctacgaaccccccacctacagccctgcagggaacctcgaactggcccccagcctggaggccccggggcctggcctccccgcataccccacggagaacttcgctagccagaccctggttcccccggcatatgccccgtaccccagccctgtgctatcagaggaggaagacttaccgttggacagccctgccctggaggtctcggacagcgagtcggatgaggccctcgtggctggccccgaggggaagggatccgaggcagggactcgcaagaagctgcgcctgtaccagttcctgctggggctactgacgcgcggggacatgcgtgagtgcgtgtggtgggtggagccaggcgccggcgtcttccagttctcctccaagcacaaggaactcctggcgcgccgctggggccagcagaaggggaaccgcaagcgcatgacctaccagaagctggcgcgcgccctccgaaactacgccaagaccggcgagatccgcaaggtcaagcgcaagctcacctaccagttcgacagcgcgctgctgcctgcagtccgccgggcctgagcacacccgaggctcccacctgcggagccgctgggggacctcacgtcccagccaggatccccctggaagaaaaagggcgtccccacactctaggtgataggacttacgcatccccaccttttggggtaaggggagtgctgccctgccataatccccaagcccagcccgggcctgtctgggattccccacttgtgcctggggtccctctgggatttctttgtcatgtacagactccctgggatcctcatgttttgggtgacaggacctatggaccactatactcggggaggcagggtagcagtgcttccagagtcccaagagcttctctgggattttcttgtgatatctgattccccagtgaggcctgggacctttttaagatcgctgtgtgtctgtaaaccctgaatctcatctggggtgggggccctgctggcaaccctgagccctgtccaaggttccctcttgtcagatctgagatttcctagttatgtctggggccctctgggagctgttatcatctcagatctcttcgcccatctatggctgtgttgtcacatctgtcccctcatttttgagatcccccaattctctggaactattctgctgcccctttttatgtgtctggagttccccaatcacatctagggctcctccaaaaaaaaaaaaaaaaaaaa

SEQ ID NO: 2 - protein, 262 aa, Homo sapiens
MLALEAAQLDGPHFSCLYPDGVFYDLDSCKHSSYPDSEGAPDSLWDWTVAPPVPATPYEAFDPAAAAFSHPQAAQLCYEPPTYSPAGNLELAPSLEAPGPGLPAYPTENFASQTLVPPAYAPYPSPVLSEEEDLPLDSPALEVSDSESDEALVAGPEGKGSEAGTRKKLRLYQFLLGLLTRGDMRECVWWVEPGAGVFQFSSKHKELLARRWGQQKGNRKRMTYQKLARALRNYAKTGEIRKVKRKLTYQFDSALLPAVRRA

SEQ ID NO: 3 - DNA, 2786 bp, Mus musculus
  CDS (19)..(822)
agcctgctctgaaccaccatgcttgctctggaggctgcacagctggatggcccacacttaagctgtttgtacccagaaggagtcttctacgacctggacagctgcaagcccttcagttacccagattcagatgggggccttgactctacatggggctggacagaggccccgcctgctcctgccatcgctccctatgaagccttcgatcctgctactgctgcctttagccactcccaaactgttcagctctgttatagccatggtcctaacccctccacctatagccccatggggaccctcgacccagcccccagcttggaggccccagggcctggcctccaggtgtaccccccagaggacttcaccagccagaccctgggctccttggcttatgctccgtaccccagccctgtgctatcagaggaagaagacattatgctggacagccccgccctggaggtctcggacagtgagtcagacgaggccctcttggctggctccgaggggaggggatctgaggcaggtgcacgcaagaagctgcgcctgtaccagttcttgctggggttgctcctacgcggggacatgcgcgagtgcgtgtggtgggtggagccaggtgccggcgtcttccagttctcctccaagcacaaggagttgttggctcgccgctggggccagcagaagggcaaccgcaagcgcatgacgtatcagaagctggcccgagcgctgcgcaactatgccaagacaggcgaaatccgcaaggtcaaacgcaaactcacctaccagtttgacagcgcgctgctgccagcctcccggcatgtctgagcactccgctaaggacccctttctggcccctaagtcccatggagccccatatgagggcagtcagggttctcagctctccctagagcctccccagagtttcctgtgccgtgtataggattccaatctaggatggtcgtgtttgagggagcactggccattctacacggtttcagaatggcaggtttctcgggggggggggggatgggggagccctgatgtcgtctacggttccagaaaccgcagttcttgcgagtcctgtgagctcacatgacatctcaccagcaggtggcgctgtctacagccccccccaaacccttgttttgttggccagataggtcggtccctctgtactccccctgaagcccttgttagatctgaggtctagttatgtttggagctctctgagaaccctgtgccacctgtgtgtgacttttctctgcgtccgtttatgacttttgtttgtttgagacagggtctcattatgtagctcaggctggccccccaacttttaacaatcgtcctgcctcggcctcctgagtgctgggatgacaagggtgcaccatcacaccaggttttttccttttttgagagattttactatgtaacccgggctgggctattctcaagctagtggcagtcctcttgcctcaggctcctcttgcctcaggcaccccttgggaccctctgggacctatgtccgagatgaatggctgggtaaggtagggtgggaggttcagtgaaccttataggttgggcccttccttctgggatcccttgatcatatgggaagttctctaggctctcagcagccctgcattcacacactgactgaggcgcgacctgtatgttgtgtttgagggggatgtgtggcagaggtatggctgtggcaaggccggtgccttttattcttgagattgagtcttatgtagctcaggctggtcttgaactcactctcactgtgtagctagggaagaccttgagcccttgcctcagccagaatgctcagatgtcaggcagcgcaccacgtgactgtttctttccattgtcttgtctttttgttgttgttgttttcgagacagggtttctctgtgtagccctggccgttctggaactcactctgtagaccaggctggccttaaactcagaaatccacctgcctctgcctctcaaatgctgggattaaaggcgtgcaccaccacgccctttttcctttttcttaagtcaaggtctatttgtgtagcccaggctggcttcaagctcatgacactcttcctgcctctgcgtctggaactatgaacataccttactactctgtgcttacccacgccatgggtagacagacttctagacttggtcaacccccacccacaaggcaggcaaattaagtccctgcaggtgctttctttggaggaaagcccgctttcatagtgatctgtcaagctagaaagcactcccgccacccagatatctaagtgtgaatctttggacaactggtgacttctggccagcttttctggggccggcctctgtccaggtgtctgcccaggaccctgctcagtgcctgtctgtctcacaccagtgacttcctcacacccgcctgttcaggcccagtcttttccgttcaagtgctacaggccaagtaggctcaaactgtgggtttgtcttgggcatccacagcagaatcagaacccagagctttgaagcctgagtgaggggaggggggcactcaggcttccgtctctctgagaaacagacgatgaagaggcccttaaaactctttgcaaccccatgagcctccccaatgagcctgtgacacaccggaactcacctctgtgggtggccgggagggggaacagggtcatggaagatccagatgtccatgtggtcaaggctaaactgtactaaataaaattatttctcatcaccactatacatgcataataaataaagtgtacatcaaaatt

SEQ ID NO: 4 - protein, 267 aa, Mus musculus
MLALEAAQLDGPHLSCLYPEGVFYDLDSCKPFSYPDSDGGLDSTWGWTEAPPAPAIAPYEAFDPATAAFSHSQTVQLCYSHGPNPSTYSPMGTLDPAPSLEAPGPGLQVYPPEDFTSQTLGSLAYAPYPSPVLSEEEDIMLDSPALEVSDSESDEALLAGSEGRGSEAGARKKLRLYQFLLGLLLRGDMRECVWWVEPGAGVFQFSSKHKELLARRWGQQKGNRKRMTYQKLARALRNYAKTGEIRKVKRKLTYQFDSALLPASRHV

SEQ ID NO: 5 - DNA, 2583 bp, Rattus norvegicus
  CDS (9)..(818)
gaaccaccatgcttgctctggaggctgcacagctggatggcccacacttaagctgtttgcagtacccagaaggtgtcttctatgacctggacagctgcaagtccttcagttacccagattcagatgggggccctgactccacatggggctggacagaggccccgcctgcccctgccatcgctgcctatgaagctttcgaccctgctgctacggcagcctttgcccacacccaggctgtgcagctctgttatggtcatggtcctagcccctccacctacagtcccgtgggaacccttgacccagcccccagcttggaggcttcggggcctggcctccaggtatacccctcagaggacttcaccagccagaccctgggctccttggcttatgctccgtaccccagccctgtgctatcagaggaggaagacattctgctggacagccctgccctggaggtctcggacagtgagtcagacgaggccctcttggctggctccgaggggaggggatctgaggcaggtgcacgcaagaagctgcgcctgtaccagttcttgctggagttactcctgcgcggggacatgcgcgagtgcgtgtggtgggtggagccaggtgctggcgtcttccagttctcctccaagcacaaggagttgttggctcgccgctggggccagcagaagggcaaccgcaaacgcatgacgtatcagaagctggcccgagcgctgcgcaactatgccaagacaggcgaaatccgcaaggtcaaacgcaagctcacctaccagtttgacagcgcgctgctgccagccgcccggcgtgcctgagcgccccgctgggacccctttctgatccctaagtcccagtgcccatagaaccccatattggggatcagcaggctgccagggtcctcagctctcaccagggcctccccagagtctcctgtgccatgtatgggattccaacccaggatggtcctgtttgagggctcactggccattctacgctgttccaaaatggcaagtttttctttgggggcccttggtgttgtctgcggttccagaatccgcagatacttcttgtgagccctgtaagctcacacgctatcgcaccagcaggtggcgctgtctacagcccccaaacccctgtatctgttggccggatgggtccctctgttctcccctgaggcccttgtcagatctgagatctagttatgtttggagctccctgagaacactgtgccacctgtgtgtgacttttctctgtatccatttatgacttttatttgtttgagacagggtctcactatgtagttcaggctggcccccaacctgtgacaatcctcctgcctcagcctcctgaatcctggggattacaaaggtgcaccatcagaccaggcattttccatttttgagagaagattttactatgtaacccaggctgtcctcgagcttgcctcagccatccctcgggtccctctgggatctatgtcccagatgaattgttgggtagggtagggatggtgtgttcggtgaatcttataggttggaccctcccttctggggtcttttgattatatgggaagttctctaggctcttagcagccctgcattcacacactgatggtcccgtttgaagtgtgatctgtgtatggtgtttgagggtcatactggcacttggccagtttgggatgtggggcagaggtatggctgtgacaaggcttcggtttttgagattgggtctcatgtatctcagtctggccttgaactccctatatagctgaggatgaccttgaacccttgcctcagccagaatgctcagattccaggcagcactatattactatttcccttttcttacgtcaaggtctgtctatgtagcccaggctagctttaatctcataacactcttcctgcctctgtgactggaaagctagggctacggacatgttttaccacactggtttagccaagcatacccctgttttatcacccttgccatggctaaacagacttctagagctggtcaccccccacccacaaggcaggcaaattaagtccctgcaggtgttttcttcagaggaaagcccccttttacagtcatctttcaaactagagagcattcccacaatccagatatctgaatgtgaatttttggacaactggtaacctctggccagcttttctggagccagcctttgtccagatgttggctcaggaccctgctcagtgcctgcctgtctcacaccagtgacttctcacaactgcccgtccgggcccagtgttttacctgctgcaggtcaagcagactcaaactgtgggcttctcttgggcctccatggcagaatcagaactcagagttttgaagcctgagtgagctgggccaccatgaagaggcccttaaaactctgccacccagatgggcagcacctcagtgggtggccagggggcaggaaaggatcatggaagatccagatgttcatgtggttaaggttaaactggtaccaaataaaattatttcttaaaacccaacatcatcaatctacatgcataataaagccctacatcaaaaaaaaaaaaaaaaaaaaaaaaa

SEQ ID NO: 6 - protein, 269 aa, Rattus norvegicus
MLALEAAQLDGPHLSCLQYPEGVFYDLDSCKSFSYPDSDGGPDSTWGWTEAPPAPAIAAYEAFDPAATAAFAHTQAVQLCYGHGPSPSTYSPVGTLDPAPSLEASGPGLQVYPSEDFTSQTLGSLAYAPYPSPVLSEEEDILLDSPALEVSDSESDEALLAGSEGRGSEAGARKKLRLYQFLLELLLRGDMRECVWWVEPGAGVFQFSSKHKELLARRWGQQKGNRKRMTYQKLARALRNYAKTGEIRKVKRKLTYQFDSALLPAARRA

SEQ ID NO: 7 - DNA, 1885 bp, Homo sapiens
  CDS (371)..(1795)
gaaactcccgcctggccaccataaaagcgccggccctccgcttccccgcgagacgaaacttcccgtcccggcggctctggcacccagggtccggcctgcgccttcccgccaggcctggacactggttcaacacctgtgacttcatgtgtgcgcgccggccacacctgcagtcacacctgtagccccctctgccaagagatccataccgaggcagcgtcggtggctacaagccctcagtccacacctgtggacacctgtgacacctggccacacgacctgtggccgcggcctggcgtctgctgcgacaggagcccttacctcccctgttataacacctgaccgccacctaactgcccctgcagaaggagcaatggccttggctcctgagagggcagccccacgcgtgctgttcggagagtggctccttggagagatcagcagcggctgctatgaggggctgcagtggctggacgaggcccgcacctgtttccgcgtgccctggaagcacttcgcgcgcaaggacctgagcgaggccgacgcgcgcatcttcaaggcctgggctgtggcccgcggcaggtggccgcctagcagcaggggaggtggcccgccccccgaggctgagactgcggagcgcgccggctggaaaaccaacttccgctgcgcactgcgcagcacgcgtcgcttcgtgatgctgcgggataactcgggggacccggccgacccgcacaaggtgtacgcgctcagccgggagctgtgctggcgagaaggcccaggcacggaccagactgaggcagaggcccccgcagctgtcccaccaccacagggtgggcccccagggccattcctggcacacacacatgctggactccaagccccaggccccctccctgccccagctggtgacaagggggacctcctgctccaggcagtgcaacagagctgcctggcagaccatctgctgacagcgtcatggggggcagatccagtcccaaccaaggctcctggagagggacaagaagggcttcccctgactggggcctgtgctggaggcgaggccgcggccccagagtccccgcaccaggcagagccgtacctgtcaccctccccaagcgcctgcaccgcggtgcaagagcccagcccaggggcgctggacgtgaccatcatgtacaagggccgcacggtgctgcagaaggtggtgggacacccgagctgcacgttcctatacggccccccagacccagctgtccgggccacagacccccagcaggtagcattccccagccctgccgagctcccggaccagaagcagctgcgctacacggaggaactgctgcggcacgtggcccctgggttgcacctggagcttcgggggccacagctgtgggcccggcgcatgggcaagtgcaaggtgtactgggaggtgggcggacccccaggctccgccagcccctccaccccagcctgcctgctgcctcggaactgtgacacccccatcttcgacttcagagtcttcttccaagagctggtggaattccgggcacggcagcgccgtggctccccacgctataccatctacctgggcttcgggcaggacctgtcagctgggaggcccaaggagaagagcctggtcctggtgaagctggaaccctggctgtgccgagtgcacctagagggcacgcagcgtgagggtgtgtcttccctggatagcagcagcctcagcctctgcctgtccagcgccaacagcctctatgacgacatcgagtgcttccttatggagctggagcagcccgcctagaacccagtctaatgagaactccagaaagctggagcagcccacctagagctggccgcggccgcccagtctaataaaaagaactccagaaca

SEQ ID NO: 8 - protein, 474 aa, Homo sapiens
MALAPERAAPRVLFGEWLLGEISSGCYEGLQWLDEARTCFRVPWKHFARKDLSEADARIFKAWAVARGRWPPSSRGGGPPPEAETAERAGWKTNFRCALRSTRRFVMLRDNSGDPADPHKVYALSRELCWREGPGTDQTEAEAPAAVPPPQGGPPGPFLAHTHAGLQAPGPLPAPAGDKGDLLLQAVQQSCLADHLLTASWGADPVPTKAPGEGQEGLPLTGACAGGEAAAPESPHQAEPYLSPSPSACTAVQEPSPGALDVTIMYKGRTVLQKVVGHPSCTFLYGPPDPAVRATDPQQVAFPSPAELPDQKQLRYTEELLRHVAPGLHLELRGPQLWARRMGKCKVYWEVGGPPGSASPSTPACLLPRNCDTPIFDFRVFFQELVEFRARQRRGSPRYTIYLGFGQDLSAGRPKEKSLVLVKLEPWLCRVHLEGTQREGVSSLDSSSLSLCLSSANSLYDDIECFLMELEQPA

SEQ ID NO: 9 - DNA, 1972 bp, Homo sapiens
  CDS (371)..(1882)
gaaactcccgcctggccaccataaaagcgccggccctccgcttccccgcgagacgaaacttcccgtcccggcggctctggcacccagggtccggcctgcgccttcccgccaggcctggacactggttcaacacctgtgacttcatgtgtgcgcgccggccacacctgcagtcacacctgtagccccctctgccaagagatccataccgaggcagcgtcggtggctacaagccctcagtccacacctgtggacacctgtgacacctggccacacgacctgtggccgcggcctggcgtctgctgcgacaggagcccttacctcccctgttataacacctgaccgccacctaactgcccctgcagaaggagcaatggccttggctcctgagagggcagccccacgcgtgctgttcggagagtggctccttggagagatcagcagcggctgctatgaggggctgcagtggctggacgaggcccgcacctgtttccgcgtgccctggaagcacttcgcgcgcaaggacctgagcgaggccgacgcgcgcatcttcaaggcctgggctgtggcccgcggcaggtggccgcctagcagcaggggaggtggcccgccccccgaggctgagactgcggagcgcgccggctggaaaaccaacttccgctgcgcactgcgcagcacgcgtcgcttcgtgatgctgcgggataactcgggggacccggccgacccgcacaaggtgtacgcgctcagccgggagctgtgctggcgagaaggcccaggcacggaccagactgaggcagaggcccccgcagctgtcccaccaccacagggtgggcccccagggccattcctggcacacacacatgctggactccaagccccaggccccctccctgccccagctggtgacaagggggacctcctgctccaggcagtgcaacagagctgcctggcagaccatctgctgacagcgtcatggggggcagatccagtcccaaccaaggctcctggagagggacaagaagggcttcccctgactggggcctgtgctggaggcccagggctccctgctggggagctgtacgggtgggcagtagagacgacccccagccccgggccccagcccgcggcactaacgacaggcgaggccgcggccccagagtccccgcaccaggcagagccgtacctgtcaccctccccaagcgcctgcaccgcggtgcaagagcccagcccaggggcgctggacgtgaccatcatgtacaagggccgcacggtgctgcagaaggtggtgggacacccgagctgcacgttcctatacggccccccagacccagctgtccgggccacagacccccagcaggtagcattccccagccctgccgagctcccggaccagaagcagctgcgctacacggaggaactgctgcggcacgtggcccctgggttgcacctggagcttcgggggccacagctgtgggcccggcgcatgggcaagtgcaaggtgtactgggaggtgggcggacccccaggctccgccagcccctccaccccagcctgcctgctgcctcggaactgtgacacccccatcttcgacttcagagtcttcttccaagagctggtggaattccgggcacggcagcgccgtggctccccacgctataccatctacctgggcttcgggcaggacctgtcagctgggaggcccaaggagaagagcctggtcctggtgaagctggaaccctggctgtgccgagtgcacctagagggcacgcagcgtgagggtgtgtcttccctggatagcagcagcctcagcctctgcctgtccagcgccaacagcctctatgacgacatcgagtgcttccttatggagctggagcagcccgcctagaacccagtctaatgagaactccagaaagctggagcagcccacctagagctggccgcggccgcccagtctaataaaaagaactccagaaca

SEQ ID NO: 10 - protein, 503 aa, Homo sapiens
MALAPERAAPRVLFGEWLLGEISSGCYEGLQWLDEARTCFRVPWKHFARKDLSEADARIFKAWAVARGRWPPSSRGGGPPPEAETAERAGWKTNFRCALRSTRRFVMLRDNSGDPADPHKVYALSRELCWREGPGTDQTEAEAPAAVPPPQGGPPGPFLAHTHAGLQAPGPLPAPAGDKGDLLLQAVQQSCLADHLLTASWGADPVPTKAPGEGQEGLPLTGACAGGPGLPAGELYGWAVETTPSPGPQPAALTTGEAAAPESPHQAEPYLSPSPSACTAVQEPSPGALDVTIMYKGRTVLQKVVGHPSCTFLYGPPDPAVRATDPQQVAFPSPAELPDQKQLRYTEELLRHVAPGLHLELRGPQLWARRMGKCKVYWEVGGPPGSASPSTPACLLPRNCDTPIFDFRVFFQELVEFRARQRRGSPRYTIYLGFGQDLSAGRPKEKSLVLVKLEPWLCRVHLEGTQREGVSSLDSSSLSLCLSSANSLYDDIECFLMELEQPA

SEQ ID NO: 11 - DNA, 2051 bp, Homo sapiens
  CDS (411)..(1961)
agggtgcgaagcgccactgtttaggtttcgctttcccgggagcctgacccgcccctgacgtcgcctttcccgtctccgcagggtccggcctgcgccttcccgccaggcctggacactggttcaacacctgtgacttcatgtgtgcgcgccggccacacctgcagtcacacctgtagccccctctgccaagagatccataccgaggcagcgtcggtggctacaagccctcagtccacacctgtggacacctgtgacacctggccacacgacctgtggccgcggcctggcgtctgctgcgacaggagcccttacctcccctgttataacacctgaccgccacctaactgcccctgcagaaggagcaatggccttggctcctgagaggtaagagcccggcccaccctctccagatgccagtccccgagcgccctgcagccggccctgactctccgcggccgggcacccgcagggcagccccacgcgtgctgttcggagagtggctccttggagagatcagcagcggctgctatgaggggctgcagtggctggacgaggcccgcacctgtttccgcgtgccctggaagcacttcgcgcgcaaggacctgagcgaggccgacgcgcgcatcttcaaggcctgggctgtggcccgcggcaggtggccgcctagcagcaggggaggtggcccgccccccgaggctgagactgcggagcgcgccggctggaaaaccaacttccgctgcgcactgcgcagcacgcgtcgcttcgtgatgctgcgggataactcgggggacccggccgacccgcacaaggtgtacgcgctcagccgggagctgtgctggcgagaaggcccaggcacggaccagactgaggcagaggcccccgcagctgtcccaccaccacagggtgggcccccagggccattcctggcacacacacatgctggactccaagccccaggccccctccctgccccagctggtgacaagggggacctcctgctccaggcagtgcaacagagctgcctggcagaccatctgctgacagcgtcatggggggcagatccagtcccaaccaaggctcctggagagggacaagaagggcttcccctgactggggcctgtgctggaggcccagggctccctgctggggagctgtacgggtgggcagtagagacgacccccagccccgggccccagcccgcggcactaacgacaggcgaggccgcggccccagagtccccgcaccaggcagagccgtacctgtcaccctccccaagcgcctgcaccgcggtgcaagagcccagcccaggggcgctggacgtgaccatcatgtacaagggccgcacggtgctgcagaaggtggtgggacacccgagctgcacgttcctatacggccccccagacccagctgtccgggccacagacccccagcaggtagcattccccagccctgccgagctcccggaccagaagcagctgcgctacacggaggaactgctgcggcacgtggcccctgggttgcacctggagcttcgggggccacagctgtgggcccggcgcatgggcaagtgcaaggtgtactgggaggtgggcggacccccaggctccgccagcccctccaccccagcctgcctgctgcctcggaactgtgacacccccatcttcgacttcagagtcttcttccaagagctggtggaattccgggcacggcagcgccgtggctccccacgctataccatctacctgggcttcgggcaggacctgtcagctgggaggcccaaggagaagagcctggtcctggtgaagctggaaccctggctgtgccgagtgcacctagagggcacgcagcgtgagggtgtgtcttccctggatagcagcagcctcagcctctgcctgtccagcgccaacagcctctatgacgacatcgagtgcttccttatggagctggagcagcccgcctagaacccagtctaatgagaactccagaaagctggagcagcccacctagagctggccgcggccgcccagtctaataaaaagaactccagaaca

SEQ ID NO: 12 - protein, 516 aa, Homo sapiens
MPVPERPAAGPDSPRPGTRRAAPRVLFGEWLLGEISSGCYEGLQWLDEARTCFRVPWKHFARKDLSEADARIFKAWAVARGRWPPSSRGGGPPPEAETAERAGWKTNFRCALRSTRRFVMLRDNSGDPADPHKVYALSRELCWREGPGTDQTEAEAPAAVPPPQGGPPGPFLAHTHAGLQAPGPLPAPAGDKGDLLLQAVQQSCLADHLLTASWGADPVPTKAPGEGQEGLPLTGACAGGPGLPAGELYGWAVETTPSPGPQPAALTTGEAAAPESPHQAEPYLSPSPSACTAVQEPSPGALDVTIMYKGRTVLQKVVGHPSCTFLYGPPDPAVRATDPQQVAFPSPAELPDQKQLRYTEELLRHVAPGLHLELRGPQLWARRMGKCKVYWEVGGPPGSASPSTPACLLPRNCDTPIFDFRVFFQELVEFRARQRRGSPRYTIYLGFGQDLSAGRPKEKSLVLVKLEPWLCRVHLEGTQREGVSSLDSSSLSLCLSSANSLYDDIECFLMELEQPA

SEQ ID NO: 13 - DNA, 1830 bp, Mus musculus
  CDS (421)..(1794)
ctccggtaccagggtccagcctgtgacagcccatccaggctcggacgctggattaacacctgtgacatcacgtgtgcgctcccgctacatctgtagtcacatctacagccaccttggcagaggaatctacactgagttctgaacctaggtgaccacaagttctcaaacctcatctgtgaccctcaacaccctaatacctgggtcacacttgtgaaactgaaattcctacctgttaccaacacttgtaacgtttaaaacacatttgtgaaacttggtcacactatctgtggctacaacctaaaaccatagaggcacccaaggggccttatttgcaatagctgactgctccagtgactacaaggcatcacagagtagtagcatctactttgattctcccagactgcctgtgtagacggagcaatggctgaagtgaggggggtccagcgagtgctgtttggagactggctattgggggaggtcagcagcggccagtacgaggggctgcagtggctgaacgaggctcgcacagtcttccgcgtaccctggaagcatttcggtcgtagggatctggatgaagaagatgcacagatcttcaaggcctgggctgtggcccgagggaggtggccacctagtggagttaacctgccacccccagaggctgaggctgctgagcgaagagagcgaagaggctggaagaccaacttccgctgtgcactccacagcacagggcgttttatcttgcgccaagacaattcaggggatccagttgatccgcataaggtgtacgaacttagccgggagcttggatctactgtgggcccagccacggaaaatagggaagaagtgagcctcagcaatgctctgcccacacagggtgtgtccccaggatcatttctggcaagagaaaatgctgggctccaaaccccaagccctctgctttctagtgatgccggggacctcttgcttcaggttctgcagtacagccacatactggaatccgagtctggggcagaccccgtcccaccacaggctcctggccaggagcaagaccgtgtttacgaggaaccctatgcagcatggcaggtggaagctgtccccagtcccaggcctcaacagccagctctcaccgagcgcagccttgggttcctggatgtgaccatcatgtacaagggccgcacagtgctacaggcagtggtggggcaccccagatgcgtgttcctgtacagccccatggccccagcagtaagaacttcagagccccagccggtgatctttcccagtcctgctgagctcccagatcagaagcagctgcactacacagagacgcttctccagcatgtgtctcccggccttcagctggagcttcgaggaccgtcactgtgggccctgcgtatgggcaagtgcaaggtgtactgggaggtaggcagccctatgggcactaccggcccctccaccccaccccagctgctggagcgcaaccgccacacccccatcttcgacttcagcactttcttccgagaactggaggagtttcgggctcggaggcggcaagggtcaccacactacaccatctacctgggttttgggcaagacttgtcagcagggaggcccaaggagaagaccctgatcctggtgaagctggagccatgggtatgcaaggcatacctggagggcgtgcagcgtgagggtgtgtcctccctggacagcagcagtctcggcttgtgcttgtctagcaccaacagtctctacgaagacatcgaacacttcctcatggacctgggtcagtggccttgactcagaatcccaactcccaataaatagttcaaaatc

SEQ ID NO: 14 - protein, 457 aa, Mus musculus
MAEVRGVQRVLFGDWLLGEVSSGQYEGLQWLNEARTVFRVPWKHFGRRDLDEEDAQIFKAWAVARGRWPPSGVNLPPPEAEAAERRERRGWKTNFRCALHSTGRFILRQDNSGDPVDPHKVYELSRELGSTVGPATENREEVSLSNALPTQGVSPGSFLARENAGLQTPSPLLSSDAGDLLLQVLQYSHILESESGADPVPPQAPGQEQDRVYEEPYAAWQVEAVPSPRPQQPALTERSLGFLDVTIMYKGRTVLQAVVGHPRCVFLYSPMAPAVRTSEPQPVIFPSPAELPDQKQLHYTETLLQHVSPGLQLELRGPSLWALRMGKCKVYWEVGSPMGTTGPSTPPQLLERNRHTPIFDFSTFFRELEEFRARRRQGSPHYTIYLGFGQDLSAGRPKEKTLILVKLEPWVCKAYLEGVQREGVSSLDSSSLGLCLSSTNSLYEDIEHFLMDLGQWP

SEQ ID NO: 15 - DNA, 10 bp, Artificial Sequence
  Spi-B binding sequence
nngaggaann

SEQ ID NO: 16 - DNA, 27 bp, Artificial Sequence
  Primer for mouse ifna4 promoter
cccccacactttacttttttgacagaa

SEQ ID NO: 17 - DNA, 27 bp, Artificial Sequence
  Primer for ifna4 promoter
tacaggttctctgagagcctgctgtgt

SEQ ID NO: 18 - DNA, 27 bp, Artificial Sequence
  Primer for mouse ifnb1 promoter
agcttgaataaaatgaatattagaagc

SEQ ID NO: 19 - DNA, 27 bp, Artificial Sequence
  Primer for mouse ifnb1 promoter
caagatgaggcaaaggctgtcaaaggc

SEQ ID NO: 20 - RNA, 21 nt, Artificial Sequence
  mouse Spi-B siRNA
agacaggcgaaauccgcaauu

SEQ ID NO: 21 - RNA, 21 nt, Artificial Sequence
  mouse Spi-B siRNA
uugcggauuucgccugucuuu

SEQ ID NO: 22 - RNA, 21 nt, Artificial Sequence
  mouse Spi-B siRNA
ugucugagcacuccgcuaauu

SEQ ID NO: 23 - RNA, 21 nt, Artificial Sequence
  mouse Spi-B siRNA
uuagcggagugcucagacauu

SEQ ID NO: 24 - RNA, 21 nt, Artificial Sequence
  mouse Spi-B siRNA
gcgcaugacguaucagaaguu

SEQ ID NO: 25 - RNA, 21 nt, Artificial Sequence
  mouse Spi-B siRNA
cuucugauacgucaugcgcuu

SEQ ID NO: 26 - RNA, 21 nt, Artificial Sequence
  mouse Spi-B siRNA
cgaccuguauguuguguuuuu

SEQ ID NO: 27 - RNA, 21 nt, Artificial Sequence
  mouse Spi-B siRNA
aaacacaacauacaggucguu

SEQ ID NO: 28 - RNA, 21 nt, Artificial Sequence
  human Spi-B siRNA
gaacuucgcuagccagaccuu

SEQ ID NO: 29 - RNA, 21 nt, Artificial Sequence
  human Spi-B siRNA
ggucuggcuagcgaaguucuu

SEQ ID NO: 30 - RNA, 21 nt, Artificial Sequence
  human Spi-B siRNA
cuggacagcugcaagcauuuu

SEQ ID NO: 31 - RNA, 21 nt, Artificial Sequence
  human Spi-B siRNA
aaugcuugcagcuguccaguu

SEQ ID NO: 32 - RNA, 21 nt, Artificial Sequence
  human Spi-B siRNA
cagauggcgucuucuaugauu

SEQ ID NO: 33 - RNA, 21 nt, Artificial Sequence
  human Spi-B siRNA
ucauagaagacgccaucuguu

SEQ ID NO: 34 - RNA, 21 nt, Artificial Sequence
  human Spi-B siRNA
gaggaagacuuaccguugguu

SEQ ID NO: 35 - RNA, 21 nt, Artificial Sequence
  human Spi-B siRNA
ccaacgguaagucuuccucuu

SEQ ID NO: 36 - DNA, 41 bp, Artificial Sequence
  primer for mouse Ly49Q
ctagcccgggctcgagccttcaaagtagaactgaagcattc

SEQ ID NO: 37 - DNA, 42 bp, Artificial Sequence
  primer for mouse Ly49Q
ccggaatgccaagcttttctgcatcaatcctgatctcatgtc

SEQ ID NO: 38 - DNA, 40 bp, Artificial Sequence
  primer for mouse Ly49Q
ctagcccgggctcgagacacttagctgcaattagcataac

SEQ ID NO: 39 - DNA, 40 bp, Artificial Sequence
  primer for mouse Ly49Q
ctagcccgggctcgagcttttcgatttggtcaaggaggag

SEQ ID NO: 40 - DNA, 40 bp, Artificial Sequence
  primer for mouse Ly49Q
ttacaaacctggagctgagccacctgagctgcacattttt

SEQ ID NO: 41 - DNA, 40 bp, Artificial Sequence
  primer for mouse Ly49Q
aaaaatgtgcagctcaggtggctcagctccaggtttgtaa

SEQ ID NO: 42 - DNA, 46 bp, Artificial Sequence
  primer for mouse Ly49Q
ctggcacaatatgttacttcttggctttgctttcagagtcaggttt

SEQ ID NO: 43 - DNA, 46 bp, Artificial Sequence
  primer for mouse Ly49Q
aaacctgactctgaaagcaaagccaagaagtaacatattgtgccag

SEQ ID NO: 44 - DNA, 50 bp, Artificial Sequence
  primer for mouse Ly49Q
tttcagagtcaggtttcattaagcaattggctcttttcgatttggtcaag

SEQ ID NO: 45 - DNA, 50 bp, Artificial Sequence
  primer for mouse Ly49Q
cttgaccaaatcgaaaagagccaattgcttaatgaaacctgactctgaaa

(19) United States
(12) Patent Application Publication
(10) Pub. No.: US 2012/0208861 A1
(43) Pub. Date: Aug. 16, 2012

(54) IFN TYPE-I PRODUCTION INHIBITOR AND METHOD FOR SCREENING FOR SAME

(75) Inventors: Tsuneyasu Kaisho, Yokohama-shi (JP); Katsuaki Hoshino, Yokohama-shi (JP); Takahiro Sugiyama, Yokohama-shi (JP)

(73) Assignee: RIKEN, Wako-shi (JP)

(21) Appl. No.: 13/061,426

(22) PCT Filed: Aug. 28, 2009
(86) PCT No.: PCT/JP2009/065105

§ 371 Date: Apr. 5, 2011

(30) Foreign Application Priority Data
Aug. 28, 2008 (JP) 2008-220193

(51) Int. Cl. A61K 31/713; C12N 15/85; C12Q 1/68; C07H 21/02

(52) U.S. Cl. 514/44 A; 536/24.5; 435/320.1; 435/6.13; 514/44 R

(57) ABSTRACT

It has been found that Spi-B, in cooperation with IRF-7, induces type I IFN production. This invention is based on the finding, and provides a type I IFN production inhibitor comprising an antisense nucleic acid or siRNA against Spi-B, or an expression vector capable of expressing the same; a screening method for a substance capable of inhibiting type I IFN production, comprising selecting a substance that suppresses the expression or function of Spi-B as a substance capable of inhibiting type I IFN production; and a type I IFN production inducer comprising an expression vector capable of expressing Spi-B and an expression vector capable of expressing IRF-7 in combination, and the like.

### TECHNICAL FIELD

The present invention relates to a type I IFN production inhibitor, a prophylactic/therapeutic agent for a disease associated with excess production of type I IFN, a method of screening for a substance capable of inhibiting type I IFN production and the like. The present invention also relates to a type I IFN production inducer and the like.

### BACKGROUND ART

Dendritic cells (DCs) sense nucleic acids through a group of pattern recognition receptors (PRRs) and produce a variety of cytokines including IL-12 or type I interferons (IFNs). Nucleic acid sensing PRRs consist of Toll-like receptors (TLRs) and RIG-I-like receptors (RLRs) (non-patent document 1). TLRs for nucleic acids are type I membrane proteins expressed in the endosome and include TLR3, TLR7, TLR8 and TLR9 (non-patent documents 2 and 3). Nucleic acid-sensing RLRs such as RIG-I and MDA5 are cytosolic proteins. DCs are heterogeneous and consist of several kinds of subsets (non-patent document 4). These DC subsets respond to PRR signaling in a subset-specific manner.

Plasmacytoid DC (pDC) is one of DC subsets that can be distinguished from conventional DC (cDC) according to the expression of many cell surface markers (non-patent document 5). Among PRRs, pDC selectively expresses TLR7 and TLR9, which sense single-stranded RNA (ssRNA) and DNA comprising the non-methylated CpG motif (CpG DNA), respectively (non-patent document 6). In response to TLR7/9 signaling, pDC can produce vast amounts of type I IFNs. This ability to produce type I IFNs, especially IFN-α, is characteristic of pDC.

Since the overproduction of type I IFNs is known to be involved in the onset and exacerbation of various autoimmune diseases (for example, systemic lupus erythematosus, Sjögren's syndrome, psoriasis, rheumatoid arthritis, multiple sclerosis and the like), inflammatory diseases, shocks (septic shock and the like), and type I IFN-related diseases such as type I diabetes, there is a demand for elucidating the mechanism behind the regulation of type I IFN production, and developing a type I IFN production inhibitor based thereon.

Interferon regulatory factor 7 (IRF-7)-deficient pDC showed severe defects in TLR7/9-induced type I IFN production (non-patent document 7) and IRF-7 expression is constitutively high in pDC (non-patent document 8), indicating that IRF-7 is a critical transcription factor for the pDC feature. Several molecules including IκB kinase α (IKKα), IRAK-1, and Osteopontin (OPN) are reported to be involved in type I IFN production by regulating IRF-7 in TLR7/9-stimulated pDC (non-patent documents 9-11). However, none of these molecules are highly expressed in pDC, and details of the mechanism behind the production of type I IFN in pDC remains unclear.

In mice lacking the IRF-8 gene, it has been reported that no generation of pDC is noted (non-patent documents 14 and 15).

Meanwhile, Spi-B is a publicly known transcription factor belonging to the Ets family (non-patent documents 12 and 13). This family consists of approximately 30 members, all of which have the DNA-binding domain similar to that of the founding member, Ets-1. This domain is called as the Ets domain and is known to bind to the purine-rich GGA(A/T) core sequence. It has been reported that knockdown of human Spi-B gene expression inhibited the generation of pDC from CD34⁺ precursor cells, indicating that Spi-B is critical for expansion or development of human pDC (non-patent document 16). However, the role of Spi-B in type I IFN gene expression has not been clarified.

### PRIOR ART REFERENCES

### Non-Patent Documents

non-patent document 1: Beutler, B. et al., Nat Rev Immunol 7, 753-766 (2007)
non-patent document 2: Medzhitov, R., Nat Rev Immunol 1, 135-145 (2001)
non-patent document 3: Takeda, K., Kaisho, T. & Akira, S., Annu Rev Immunol 21, 335-376 (2003)
non-patent document 4: Shortman, K. & Liu, Y. J. Mouse and human dendritic cell subtypes. Nature Rev Immunol 2, 151-161 (2002)
non-patent document 5: Liu, Y. J., Annu Rev Immunol 23, 275-306 (2005)
non-patent document 6: Gilliet, M., Cao, W. & Liu, Y. J., Nat Rev Immunol 8, 594-606 (2008)
non-patent document 7: Honda, K. et al., Nature 434, 772-777 (2005)
non-patent document 8: Izaguirre, A. et al., J Leukoc Biol 74, 1125-1138 (2003)
non-patent document 9: Hoshino, K. et al., Nature 440, 949-953 (2006)
non-patent document 10: Uematsu, S. et al., J Exp Med., 201, 915-923 (2005)
non-patent document 11: Shinohara, M. L. et al., Nat Immunol 7, 498-506 (2006)
non-patent document 12: Sharrocks, A. D., Nat Rev Mol Cell Biol 2, 827-37 (2001)
non-patent document 13: Oikawa, T. & Yamada, T., Gene 303, 11-34 (2003)
non-patent document 14: Schiavoni G et al., J Exp Med., 196, 1415-1425 (2002)
non-patent document 15: Tsujimura H et al., J. Immunol., 170, 1131-1135 (2003)
non-patent document 16: Schotte, R., Nagasawa, M., Weijer, K., Spits, H. & Blom, B., J Exp Med., 200, 1503-1509 (2004)

### SUMMARY OF THE INVENTION

### Problems to Be Solved by the Invention

It is an object of the present invention to elucidate the mechanism behind the regulation of type I IFN production, and to provide a type I IFN production regulator and a method of screening for a type I IFN production inhibitor based thereon.

### Means of Solving the Problems

To understand the molecular mechanisms to regulate pDC function, the present inventors first identified a group of genes expressed abundantly in pDC by DNA microarray analysis. Among these genes, the present inventors have focused a transcription factor, Spi-B. Spi-B expression transactivated the IFN-α and IFN-β promoter in synergy with IRF-7 expression, but did not transactivate the IFN-α and IFN-β promoter in synergy with IRF-1, IRF-3 or IRF-5 expression. The expression of Spi-B also exhibited slight synergistic activation with IRF-8 expression on IFN-α and IFN-β promoters, which activation, however, was much weaker than the synergistic activation with the expression of IRF-7. Hence, Spi-B synergistically activated type I IFN promoters, with selectivity for IRF-7 in the IRF family (IRF-1, 3, 5, 7, 8). The Spi-B effect was abrogated by cotransfecting Spi-B-targeting siRNA. Spi-B-deficient mice showed severe defects in in vitro and in vivo pDC responses against TLR7 and TLR9 signaling. From these results, it was found that Spi-B plays critical roles in type I IFN production of pDC through the cooperation with IRF-7.

The present invention has been completed based on of these findings.

Accordingly, the present invention relates to the following:

[1] A type I IFN production inhibitor comprising an antisense nucleic acid or siRNA against Spi-B, or an expression vector capable of expressing the same.
[2] A prophylactic/therapeutic agent for a disease associated with excess production of type I IFN, comprising an antisense nucleic acid or siRNA against Spi-B, or an expression vector capable of expressing the same.
[3] A method of screening for a substance capable of inhibiting type I IFN production, comprising evaluating whether a test substance suppresses the expression or function of Spi-B, and selecting a substance that suppresses the is expression or function of Spi-B as a substance capable of inhibiting type I IFN production.
[4] A type I IFN production inducer comprising an expression vector capable of expressing Spi-B and an expression vector capable of expressing IRF-7 in combination.
[5] An antisense nucleic acid or siRNA against Spi-B, or an expression vector capable of expressing the same, to be used to inhibit type I IFN production.
[6] An antisense nucleic acid or siRNA against Spi-B, or an expression vector capable of expressing the same, to be used to prevent or treat a disease associated with excess production of type I IFN.
[7] A combination comprising an expression vector capable of expressing Spi-B and an expression vector capable of expressing IRF-7, to be used to induce type I IFN production.
[8] A method of inhibiting type I IFN production in a mammal, comprising administering to the mammal an effective amount of an antisense nucleic acid or siRNA against Spi-B, or an expression vector capable of expressing the same.
[9] A method of preventing or treating a disease associated with excess production of type I IFN in a mammal, comprising administering to the mammal an effective amount of an antisense nucleic acid or siRNA against Spi-B, or an expression vector capable of expressing the same.
[10] A method of inducing type I IFN production in a mammal, comprising administering to the mammal an effective amount of an expression vector capable of expressing Spi-B and an effective amount of an expression vector capable of expressing IRF-7 in combination.

### Effect of the Invention

The type I IFN production inhibitor of the present invention is capable of potently inhibiting type I IFN production on the basis of the novel mechanism of suppression of Spi-B, and is useful as a prophylactic/therapeutic agent for various autoimmune diseases (for example, systemic lupus erythematosus, Sjögren's syndrome, psoriasis, chronic rheumatoid arthritis, multiple sclerosis and the like), inflammatory diseases, shocks (septic shock and the like), and type I IFN-related diseases such as type I diabetes.

The screening method of the present invention is useful in developing a type I IFN production inhibitor based on the novel mechanism of suppression of Spi-B.

The type I IFN production inducer of the present invention has been developed on the basis of the mechanism behind the induction of type I IFN production in pDC, which reflects a synergistic effect of Spi-B and IRF-7, and is useful as a pharmaceutical such as an antitumor agent and a research tool for analyzing the mechanism behind type I IFN production.

### BRIEF DESCRIPTION OF THE DRAWINGS

FIG. 1 shows an analysis by RT-PCR of the expression of Spi-b in DC. CD24: CD24^{high}CD11b^{low}cDC, CD11b: CD24^{low}CD11b^{high} cDC, GMDC: cDC induced with GM-CSF.

FIG. 2 shows an evaluation of IFN-α (A) and IFN-β (B) promoter activity by luciferase assay. A comparison of IRF-1, -3, -5, and -7.

FIG. 3 shows an evaluation of IFN-α and IFN-β promoter activity by luciferase assay. A comparison of IRF-7 and IRF-8.

FIG. 4 shows the suppression of IFN-β promoter activity by an Spi-B-targeting siRNA.

FIG. 5 shows the detection of pDC in the spleen of a wild-type or Spi-b-deficient mouse.

FIG. 6 shows cytokine production in the bone marrow pDC of a wild-type or Spi-b-deficient mouse.

FIG. 7 shows changes in serum cytokine concentrations in wild-type or Spi-b-deficient mice after injection of poly-U RNA.

FIG. 8 shows the suppression of human IFN-β promoter activity by a human Spi-B-targeting siRNA.

FIG. 9 shows the association of Spi-B and IRF-7. 293 cells were allowed to express HA-tagged Spi-B or a FLAG-tagged IRF family member, and the cell extract, or the immunoprecipitate from the cell extract, was analyzed by SDS-PAGE-based electrophoresis and immunoblotting.

FIG. 10 shows a FACS analysis of bone marrow and spleen cells derived from a wild-type mouse and an Spi-B-deficient mouse. Each numerical figure is a % value.

FIG. 11 shows an analysis of the Ly49Q gene promoter. Activation of a 3698 bp region by Spi-B and IRF family members.

FIG. 12 shows an analysis of Ly49Q gene promoters having various deletions.

FIG. 13 shows an analysis of Ly49Q gene promoters having various deletions.

FIG. 14 shows an analysis of Ly49Q gene promoters incorporating a mutation at three putative Ets family transcription factor-binding sites.

### MODES FOR EMBODYING THE INVENTION

### 1. Type I IFN Production Inhibitor

The present invention provides a type I IFN production inhibitor comprising a substance that inhibits the expression or function of Spi-B.

Spi-B is a publicly known transcription factor belonging to the Ets family. This family consists of about 30 members, all of which have a DNA-binding domain similar to that of the founding member Ets-1. This domain is called the Ets domain, and is known to bind to the purine-rich GGA (A/T) core sequence. The Spi-B used in the present invention is derived from a mammal. Examples of the mammal include, but are not limited to, laboratory animals such as mice, rats, hamsters, guinea pigs, and other rodents, and rabbits; domestic animals such as swines, bovines, goat, horses, sheep, and minks; companion animals such as dogs and cats; and primates such as humans, monkeys, cynomolgus monkeys, rhesus monkeys, marmosets, orangutans, and chimpanzees. Representative nucleotide sequences and amino acid sequences of human, mouse and rat Spi-B are registered with the GenBank as follows:

### [Human Spi-B]

Nucleotide sequence (cDNA sequence): Accession number
NM_{—}003121 (version NM_{—}003121.2) (SEQ ID NO:1)
Amino acid sequence: Accession number NP_{—}003112 (version NP_{—}003112.2) (SEQ ID NO:2)

### [Mouse Spi-B]

Nucleotide sequence (cDNA sequence): Accession number NM_{—}019866 (version NM_{—}019866.1) (SEQ ID NO:3)
Amino acid sequence: Accession number NP_{—}063919 (version NP_{—}063919.1) (SEQ ID NO:4)

### [Rat Spi-B]

Nucleotide sequence (cDNA sequence): Accession number NM_{—}001024286 (version NM_{—}001024286.1) (SEQ ID NO:5)
Amino acid sequence: Accession number NP_{—}001019457 (version NP_{—}001019457.1) (SEQ ID NO:6)

Type I IFNs include IFN-α and IFN-β. As shown in an Example below, Spi-B promotes the transcription of IFN-α and β in cooperation with IRF-7. Therefore, by inhibiting the expression or function of Spi-B, the production of IFN-α or IFN-β can be suppressed.

Type I IFNs are produced by a wide variety of cells. Examples of cells that produce type I IFNs include dendritic cells, lymphocytes (T cells, B cells), macrophages, fibroblasts, vascular endothelial cells, osteoblasts and the like. Dendritic cells include plasmacytoid dendritic cells (pDC), conventional dendritic cells (cDC) and the like, and can be classified by the expression of cell surface markers and the like. pDC can be identified as dendritic cells that are positive for B220 and PDCA-1. The inhibitor of the present invention inhibits type I IFN production in various cells; although the type of the cell is not particularly limited, the expression of Spi-B is high in dendritic cells, particularly in pDC, so that the inhibitor of the present invention is advantageous in inhibiting type I IFN production in dendritic cells, particularly in pDC. Because pDC possesses a potent capability of IFNα production, the inhibitor of the present invention is particularly advantageous in inhibiting the production of IFN-α in pDC.

Type I IFNs are produced in response to various stimuli. The stimuli include TLR7 ligands, TLR9 ligands, TLR3 ligands, RIG-I ligands, MDA5 ligands, double-stranded DNAs (receptors of double-stranded DNAs are reportedly DAI (DLM-1/ZBP1) and unknown receptors (Nature. 2007, 448:501-5)) and the like. TLR7 ligands include ssRNA, poly-U RNA, imidazoquinoline derivatives and the like. TLR9 ligands include non-methylated CpG DNA and the like. TLR3 ligands, RIG-I ligands, and MDA5 ligands include double-stranded RNAs and the like. RIG-I ligands include 5′-triphosphate RNAs and the like. The inhibitor of the present invention inhibits the production of type I IFNs produced in response to various stimuli, the stimuli are not particularly limited; because Spi-B promotes type I IFN production in cooperation with IRF-7, and also because IRF-7 is profoundly involved in type I IFN production via TLR7 or 9, the inhibitor of the present invention is advantageous in inhibiting type I IFN production by stimulation via TLR7 or 9.

Substances that inhibit the expression or function of Spi-B include antisense nucleic acids and siRNAs against Spi-B (i.e., antisense nucleic acid and siRNAs that specifically inhibit the expression of Spi-B), expression vectors capable of expressing the antisense nucleic acid or siRNA and the like. The antisense nucleic acids and siRNAs used in the present invention are capable of suppressing the transcription or translation of Spi-B.

An “antisense nucleic acid” refers to a nucleic acid that comprises a nucleotide sequence capable of hybridizing with a target mRNA (mature mRNA or initial transcription product) under physiological conditions for cells that express the target mRNA, and that is capable of inhibiting the translation of the polypeptide encoded by the target mRNA while in a hybridized state. The kind of the antisense nucleic acid may be DNA or RNA, or a DNA/RNA chimera. Because a natural type antisense nucleic acid easily undergoes degradation of the phosphodiester bond thereof by a nucleic acid decomposing enzyme present in the cells, the antisense nucleic acid of the present invention can also be synthesized using a modified nucleotide of the thiophosphate type (P═O in phosphate bond replaced with P=S), 2′-O-methyl type and the like, which are stable to decomposing enzymes. Other important factors for the designing of antisense nucleic acids include increases in water-solubility and cell membrane permeability and the like; these can also be cleared by choosing appropriate dosage forms such as those using liposome or microspheres.

The length of the portion that hybridizes with the target mRNA in the antisense nucleic acid is not particularly limited, as far as the portion is capable of specifically hybridizing with the mature mRNA or initial transcription product of Spi-B, and inhibiting the translation of the Spi-B polypeptide while in a hybridized state; the length is about 15 bases for the shortest and the same as the full-length sequence of the mRNA (mature mRNA or initial transcription product) for the longest. Taking into account the specificity of the hybridization, the length of the portion that hybridizes with the target mRNA is, for example, about 15 bases or more, preferably about 18 bases or more, more preferably about 20 bases or more. Taking into account the issues of the ease of synthesis, antigenicity and the like, the length of the portion that hybridizes with the target mRNA is, for example, about 200 bases or less, preferably about 50 bases or less, more preferably about 30 bases or less. Hence, the length of the portion that hybridizes with the target mRNA is, for example, about 15 to about 200 bases, preferably about 18 to about 50 bases, more preferably about 20 to about 30 bases.

The target nucleotide sequence for the antisense nucleic acid is not particularly limited, as far as it is a sequence such that the translation of Spi-B is inhibited as the antisense nucleic acid hybridizes therewith; the sequence may be the full-length sequence or a partial sequence (for example, about 15 bases or more, preferably about 18 bases or more, more preferably about 20 bases or more) of the mRNA (mature mRNA or initial transcription product) of Spi-B, or the intron portion of the initial transcription product; however, when using an oligonucleotide as the antisense nucleic acid, the target sequence is desirably located from the 5′ end of the mRNA of Spi-B to the C-terminus of the coding region.

The nucleotide sequence of the portion of the antisense nucleic acid that hybridizes with the target mRNA varies depending on the base composition of the target sequence, and has an identity of normally about 90% or more (preferably 95% or more, most preferably 100%) relative to the complementary sequence for the target sequence to ensure hybridization with the mRNA of Spi-B under physiological conditions. Nucleotide sequence identity can, for example, be calculated using the homology calculation algorithm NCBI BLAST-2 (National Center for Biotechnology Information Basic Local Alignment Search Tool) under the following conditions (gap open=5; gap extension=2; x_dropoff=50; expectancy=10; filtering=ON).

The size of the antisense nucleic acid is normally about 15 bases or more, preferably about 18 bases or more, more preferably about 20 bases or more. In view of the issues of the ease of synthesis, antigenicity and the like, the size is normally about 200 bases or less, preferably about 50 bases or less, more preferably about 30 bases or less.

Furthermore, the antisense nucleic acid may be one that not only hybridizes with the mRNA or initial transcription product of Spi-B to inhibit the translation thereof, but also is capable of binding to the Spi-B gene, which is a double-stranded DNA, to form a triple strand (triplex) and inhibit the transcription into mRNA. The antisense nucleic acid is normally single-stranded.

Antisense nucleic acids that can be used in the present invention include a polynucleotide (DNA or RNA) comprising the nucleotide sequence of the mRNA (mature mRNA or initial transcription product) that encodes Spi-B or a nucleotide sequence complementary to a partial sequence thereof with 15 bases or more. Here, the nucleotide sequence of the mRNA that encodes Spi-B includes the nucleotide sequence shown by SEQ ID NO:1, 3 or 5 and the coding region thereof.

The siRNA against Spi-B is a double-stranded RNA comprising the nucleotide sequence of the mRNA (mature mRNA or initial transcription product) that encodes Spi-B or a nucleotide sequence that is complementary to a partial sequence thereof (preferably within the coding region) (in case of the initial transcription product, intron portion is included). Transferring a short double-stranded RNA to a cell results in the degradation of mRNAs that are complementary to the RNA. This phenomenon, known as RNA interference (RNAi), has long been known to occur in nematodes, insects, plants and the like; recently, this phenomenon was confirmed as occurring also in animal cells [Nature, 411(6836): 494-498 (2001)], and this is attracting attention as a substitute technique for ribozyme.

A representative siRNA is a double-stranded oligo-RNA consisting of an RNA having a nucleotide sequence complementary to the nucleotide sequence of the mRNA of the target gene or a partial sequence thereof (hereinafter, target nucleotide sequence) and a complementary strand for the same. A single-stranded RNA wherein a sequence complementary to the target nucleotide sequence (first sequence) and a complementary sequence for the same (second sequence) are linked via a hairpin loop portion, and wherein the first sequence forms a double-stranded structure with the second sequence by assuming a hairpin loop form structure (small hairpin RNA: shRNA), also represents a preferred embodiment of siRNA.

The length of the portion complementary to the target nucleotide sequence, contained in the siRNA, is normally about 15 bases or more, preferably 18 bases or more, more preferably 20 bases or more (typically about 21 to 23 bases long), but this is not particularly limited, as far as the complementary portion can cause RNA interference. If the siRNA is longer than 23 bases, the siRNA can undergo degradation in cells to produce an siRNA having about 20 bases in length; therefore, theoretically, the upper limit of the portion complementary to the target nucleotide sequence is the full length of the nucleotide sequence of the mRNA (mature mRNA or initial transcription product) of the target gene. Taking into account the issues of the ease of synthesis, antigenicity and the like, however, the length of the complementary portion is, for example, about 200 bases or less, preferably about 50 bases or less, more preferably about 30 bases or less. Hence, the length of the complementary portion is, for example, about 15 bases or more, preferably about 18 to about 200 bases, more preferably about 20 to about 50 bases, still more preferably about 20 to about 30 bases.

Also, the full length of the siRNA is normally about 18 bases or more, for example, about 20 bases or so (typically about 21 to 23 bases long), but this is not particularly limited, as far as the siRNA can cause RNA interference, and theoretically there is no upper limit on the length of the siRNA. Taking into account the issues of the ease of synthesis, antigenicity and the like, however, the length of the siRNA is, for example, about 200 bases or less, preferably about 50 bases or less, more preferably about 30 bases or less. Hence, the length of the siRNA is, for example, about 18 bases or more, preferably about 18 to about 200 bases, more preferably about 20 to about 50 bases, still more preferably about 20 to about 30 bases. Note that the length of an shRNA is shown as the length of the double-stranded portion when it assumes a double-stranded structure.

It is preferable that the target nucleotide sequence and the sequence complementary thereto contained in the siRNA be completely complementary to each other. However, in the presence of a base mutation at a position apart from the center of the siRNA (can be fall in the range of identity of at least 90% or more, preferably 95% or more), the cleavage activity by RNA interference is not completely lost, but a partial activity can remain. On the other hand, a base mutation in the center of the siRNA has a major influence to the extent that can extremely reduce the mRNA cleavage activity by RNA interference.

The siRNA may have an additional base that does not form a base pair at the 5′- or 3′-terminal. The length of the additional base is generally 5 bases or less. Although the additional base may be a DNA or an RNA, use of a DNA improves the stability of the siRNA. Examples of the sequences of such additional bases include, but are not limited to, the sequences ug-3′, uu-3′, tg-3′, tt-3′, ggg-3′, guuu-3′, gttt-3′, ttttt-3′, uuuuu-3′ and the like.

The length of the loop portion of the hairpin loop of the shRNA is not particularly limited, as far as the loop portion can cause RNA interference, but the length is normally about 5 to 25 bases. The nucleotide sequence of the loop portion is not particularly limited, as far as a loop can be formed, and the shRNA can cause RNA interference.

The above-described antisense nucleic acid and siRNA against Spi-B can be prepared by determining the target sequence on the basis of the mRNA sequence that encodes Spi-B (for example, the nucleotide sequence shown by SEQ ID NO:1, 3 or 5, the coding region thereof) or chromosomal DNA sequence, and synthesizing a nucleotide sequence complementary thereto using a commercially available automated DNA/RNA synthesizer (Applied Biosystems, Beckman and the like). The siRNA can be prepared by separately synthesizing a sense strand and an antisense strand using an automated DNA/RNA synthesizer, and denaturing the strands in an appropriate annealing buffer solution at about 90° C. to about 95° C. for about 1 minute, and then performing annealing at about 30° C. to 70° C. for about 1 to about 8 hours. A longer double-stranded polynucleotide can be prepared by synthesizing complementary oligonucleotide strands in a way such that they overlap with each other, annealing the strands, and then performing ligation with a ligase.

In the vector capable of expressing the antisense nucleic acid or siRNA against Spi-B, these polynucleotides or the nucleic acids that encode the same (preferably DNA) are operably linked to a promoter capable of exhibiting promoter activity in cells (for example, pDC) of a recipient mammal (preferably a human or a mouse). The vector is capable of expressing the antisense nucleic acid or siRNA against Spi-B under the control of the promoter.

Any promoter capable of functioning in the cells of the recipient mammal can be used. Useful promoters include pol I promoters, pol II promoters, pol III promoters and the like. Specifically, viral promoters such as the SV40-derived initial promoter and cytomegalovirus LTR, mammalian constitutive protein gene promoters such as the β-actin gene promoter, RNA promoters such as the tRNA promoter, and the like are used.

When the expression of an siRNA is intended, it is preferable that a pol III promoter be used as the promoter. Examples of the pol III promoter include the U6 promoter, H1 promoter, tRNA promoter and the like.

The expression vector of the present invention preferably contains a transcription termination signal, i.e., a terminator region, downstream of the above-described polynucleotide or nucleic acid that encodes the same. Further more, a selection marker gene for selection of transformed cells (genes that confer resistance to drugs such as tetracycline, ampicillin, and kanamycin, genes that compensate for auxotrophic mutations, and the like) can further be contained.

Although there is no limitation on the choice of the vector to be used as the expression vector, suitable vectors for administration to mammals such as humans include viral vectors such as retrovirus, adenovirus, and adeno-associated virus. Adenovirus, in particular, has advantages such as very high gene transfer efficiency and transferability to non-dividing cells. Because the integration of transgenes into host chromosome is extremely rare, however, the gene expression is transient and generally persists only for about 4 weeks. Considering the persistence of therapeutic effect, it is also preferable to use adeno-associated virus, which offers a relatively high efficiency of gene transfer, which can be transferred to non-dividing cells as well, and which can be integrated into chromosomes via an inverted terminal repeat (ITR).

The inhibitor of the present invention is administered intravenously, intra-arterially, subcutaneously, intradermally, intramuscularly, intraperitoneally and the like in the form of an injection and the like in vivo. If the production of a neutralizing antibody against the viral vector is problematic, the adverse influence of the presence of the antibody can be lessened by topically injecting the vector in the vicinity of the affected site (in situ method).

The inhibitor of the present invention can contain, in addition to a substance that inhibits the expression or function of Spi-B, an optionally chosen carrier, for example, a pharmaceutically acceptable carrier.

Examples of the pharmaceutically acceptable carrier include, but are not limited to, excipients such as sucrose and starch; binders such as cellulose and methylcellulose; disintegrants such as starch and carboxymethylcellulose; lubricants such as magnesium stearate and Aerosil; flavoring agents such as citric acid and menthol; preservatives such as sodium benzoate and sodium hydrogen sulfite; stabilizers such as citric acid and sodium citrate; suspending agents such as methylcellulose and polyvinylpyrrolidone; dispersing agents such as surfactants; diluents such as water and physiological saline; base waxes; and the like.

To promote the introduction of a polynucleotide into a cell, the inhibitor of the present invention may further contain a reagent for nucleic acid introduction. When the polynucleotide is incorporated in a viral vector, particularly in a retroviral vector, etronectin, fibronectin, polybrene or the like can be used as a reagent for gene transfer. When the polynucleotide is incorporated in a plasmid vector, a cationic lipid such as lipofectin, lipfectamine, DOGS (transfectam), DOPE, DOTAP, DDAB, DHDEAB, HDEAB, polybrene, or poly(ethyleneimine) (PEI) can be used.

Preparations suitable for oral administration include liquids, capsules, sachets, tablets, suspensions, emulsions and the like.

Preparations suitable for parenteral administration (for example, subcutaneous injection, intramuscular injection, topical injection, intraperitoneal administration and the like) include aqueous and non-aqueous isotonic sterile injectable liquids, which may contain an antioxidant, a buffer solution, a bacteriostatic agent, an isotonizing agent and the like. Aqueous and non-aqueous sterile suspensions can also be mentioned, which may contain a suspending agent, a solubilizer, a thickening agent, a stabilizer, an antiseptic and the like. These preparations can be encapsulated in containers such as ampoules and vials for unit dosage or a plurality of dosages. It is also possible to freeze-dry the active ingredient and a pharmaceutically acceptable carrier, and store the preparation in a state that may be dissolved or suspended in an appropriate sterile vehicle just before use.

The content amount of the substance that inhibits the expression or function of Spi-B in the pharmaceutical composition is, for example, about 0.1% to 100% by weight of the entire pharmaceutical composition.

Although the dosage of an inhibitor of the present invention varies depending on the choice or activity of the active ingredient, seriousness of illness, recipient animal species, the recipient's drug tolerance, body weight, age, and the like, and cannot be generalized, the dosage is generally about 0.001 to about 500 mg/kg, based on the active ingredient, per day for an adult.

The inhibitor of the present invention is preferably safely administered to a mammal (e.g., rat, mouse, guinea pig, rabbit, sheep, horse, swine, bovine, monkey, human) so that the active ingredient substance that inhibits the expression or function of Spi-B is delivered to type I IFN-producing cells (for example, pDC).

Because the inhibitor of the present invention is capable of suppressing the expression of type I IFN genes in various cells, particularly in dendritic cells (for example, pDC), to potently inhibit type I IFN production, it is useful as a prophylactic/therapeutic agent for a disease associated with excess production of type I IFN in these cells. Diseases associated with excess production of type I IFN include various autoimmune diseases whose pathogenesis is reportedly involved by type I IFN production, and which are accompanied by anti-nucleic acid antibody production and the like (for example, systemic lupus erythematosus, Sjögren's syndrome, psoriasis, chronic rheumatoid arthritis, multiple sclerosis, scleroderma, polymyositis, periarteritis nodosa, necrotizing vasculitis, dermatomyositis, type I diabetes and the like); various inflammatory conditions and cancerous diseases in which a large number of cells die, and which are accompanied by nucleic acid leakage and the like, for example, lung disorders with inflammation (asthma, bronchitis and the like), gastrointestinal conditions with inflammation (Crohn disease, ulcerative colitis and the like), graft rejection, inflammatory chronic renal conditions (glomerulonephritis, lupus nephritis and the like), autoimmune hematologic diseases (hemolytic anemia, pure red cell anemia, sudden (toppatsusei) thrombocytopenia, aplastic anemia and the like), Hashimoto disease, contact dermatitis, Kawasaki disease, diseases involved by type I allergic reactions (allergic asthma, atopic dermatitis and the like), shocks (septic shock, anaphylactic shock, adult respiratory distress syndrome and the like), sarcoidosis, Wegener granulomatosis, Hodgkin disease, and cancers (lung cancer, gastric cancer, colon cancer, liver cancer and the like); inflammations caused by various microorganisms, for example, acute (for example, influenza virus, herpes simplex virus, vesicular stomatitis virus and the like) or chronic (for example, hepatitis B virus, hepatitis C virus and the like) inflammations caused by various viruses, inflammations caused by various bacteria, fungi or parasites; and the like.

As stated above, the inhibitor of the present invention is advantageous in inhibiting the production of type I IFNs produced in response to simulation via TLR7 or 9; therefore, the inhibitor is excellently effective in preventing or treating diseases associated with excess production of type I IFN due to stimulation via TLR7 or 9, out of the above-described diseases. Such diseases include, in particular, various autoimmune diseases whose pathogenesis is reportedly involved by type I IFN production, and which are accompanied by anti-nucleic acid antibody production and the like (for example, systemic lupus erythematosus, Sjögren's syndrome, psoriasis, chronic rheumatoid arthritis, multiple sclerosis, scleroderma, polymyositis, periarteritis nodosa, necrotizing vasculitis, delmatomyositis, type I diabetes and the like) and the like. It is known that in autoimmune diseases, a self-nucleic acid stabilizes itself and becomes capable of activating TLR7/9 when forming a complex with an autoantibody against the nucleic acid or with a DNA-binding such as LL37 or HMGB1.

The inhibitor of the present invention is useful not only for the above-described in vivo use applications, but also as a reagent for research concerning type I IFN production in vitro.

### 2. Screening Method for a Substance Capable of Inhibiting Type I IFN Production

The present invention provides a screening method for a substance capable of inhibiting type I IFN production, comprising evaluating whether a test substance suppresses the expression or function of Spi-B, and selecting a substance that suppresses the expression or function of Spi-B as a substance capable of inhibiting type I IFN production.

The test substance subjected to the screening method of the present invention may be any commonly known compound or a novel compound; examples include nucleic acids, sugars, lipids, proteins, peptides, organic low molecular compounds, compound libraries prepared using combinatorial chemistry technology, random peptide libraries, or naturally occurring ingredients derived from microorganisms, animals, plants, marine organisms and the like, and the like.

For example, when selecting a substance capable of suppressing the expression of Spi-B, a test substance and cells permitting a measurement of the expression of Spi-B are brought into contact with each other, the amount of Spi-B expressed in the cells contacted with the test substance is measured, and the amount expressed is compared with the amount of Spi-B expressed in control cells not contacted with the test substance.

A cell permitting a measurement of the expression of Spi-B refers to a cell permitting a direct or indirect evaluation of the expression level of a product, for example, the transcription product or translation product, of the Spi-B gene. The cell permitting a direct evaluation of the expression level of a product of the Spi-B gene can be a cell capable of expressing Spi-B in nature, whereas the cell permitting an indirect evaluation of the expression level of a product of the Spi-B gene can be a cell permitting a reporter assay of the transcription regulatory region of the Spi-B gene. The cell permitting a measurement of the expression of Spi-B can be a cell of the above-described mammals.

The cell capable of expressing Spi-B in nature is not particularly limited, as far as the cell potentially expresses Spi-B. Such cells can be easily identified by those skilled in the art; useful cells include primary culture cells, cell lines induced from the primary culture cells, commercially available cell lines, cell lines that can be obtained from cell banks, and the like. Cells expressing Spi-B include dendritic cells (preferably pDC) and the like.

The cells permitting a reporter assay for the transcriptional regulatory region of the Spi-B gene are cells comprising the transcriptional regulatory region of the Spi-B gene and a reporter gene operably linked to the region. The transcriptional regulatory region of the Spi-B gene and the reporter gene can be inserted into an expression vector. The transcriptional regulatory region of the Spi-B gene is not particularly limited, as far as the region is capable of regulating the expression of Spi-B gene; examples include a region between the transcription initiation site and about 2 kbp upstream thereof, a region consisting of a base sequence resulting from deletion, substitution or addition of 1 or more bases in the base sequence of the region, and having the capability of regulating the transcription of the Spi-B, and the like. The reporter gene may be any gene that encodes a detectable protein or an enzyme that catalyzes the production of a detectable substance; examples include the GFP (green fluorescent protein) gene, GUS (β-glucuronidase) gene, LUC (luciferase) gene, CAT (chloramphenicol acetyltransferase) gene and the like.

The cells to which the transcriptional regulatory region of the Spi-B gene and a reporter gene operably linked to the region are introduced are not particularly limited, as far as the regulatory function for the transcription of the Spi-B gene can be evaluated, i.e., as far as the amount of the reporter gene expressed can be quantitatively analyzed. However, it is preferable that the cells used for the gene transfer be capable of expressing the Spi-B gene in nature (for example, dendritic cells, preferably pDC) since they express a physiological transcription regulatory factor for the Spi-B gene and are thought to be more appropriate for the evaluation of the expression regulation of the Spi-B gene.

Contact of the test substance with the cells permitting a measurement of the expression of Spi-B can be performed in an appropriate culture medium. The culture medium is chosen as appropriate according to the choice of cells used and the like; examples include minimal essential medium (MEM) Dulbecco's modified minimal essential medium (DMEM), RPMI1640 medium, 199 medium and the like containing about 5% to 20% fetal bovine serum. Cultivation conditions are also determined as appropriate according to the choice of cells used and the like; for example, the pH of the medium is about 6 to about 8, cultivation temperature is generally about 30° C. to about 40° C., and cultivation time is about 12 to about 72 hours.

Next, the amount of Spi-B expressed in the cells contacted with the test substance is measured. A measurement of the amount expressed can be performed by a method known per se in view of the choice of cells used and the like. For example, when using cells capable of expressing Spi-B in nature as the cells permitting a measurement of the expression of Spi-B, the amount expressed of a product of the Spi-B gene, for example, the transcription product (mRNA) or translation product (polypeptide), can be measured by a method known per se. For example, the amount of transcription product expressed can be measured by preparing total RNA from the cells, and performing RT-PCR, Northern blotting and the like.

The amount of translation product expressed can also be measured by preparing an extract from the cells, and performing an immunological technique. Immunological techniques that can be used include radioimmunoassay method (RIA method), ELISA method (Methods in Enzymol. 70: 419-439 (1980)), fluorescent antibody method, Western blotting method and the like. Meanwhile, when using cells permitting a reporter assay of the transcription regulatory region of the Spi-B gene as the cells permitting a measurement of the expression of Spi-B, the amount expressed can be measured on the basis of the signal intensity of a reporter.

Subsequently, the amount of Spi-B expressed in the cells contacted with the test substance is compared with the amount of Spi-B expressed in control cells not contacted with the test substance. This comparison of the amounts expressed is preferably performed on the basis of the presence or absence of a significant difference. Although the amount of Spi-B expressed in the control cells not contacted with the test substance may be measured before or simultaneously with the measurement of the amount of Spi-B expressed in the cells contacted with the test substance, it is preferable, from the viewpoint of experimental accuracy and reproducibility, that the amount of Spi-B expressed in the control cells be a simultaneously measured.

A substance judged as a result of the comparison to suppress the expression of Spi-B can be selected as a substance capable of inhibiting type I IFN production.

When selecting a substance capable of suppressing the function of Spi-BS, the function (activity) of Spi-B is measured in the presence of a test substance, and comparing the function (activity) with the function (activity) of Spi-B in the absence of the test substance.

Functions of Spi-B include binding to DNA having a purine-rich GGA (A/T) core sequence (for example, 5′-GAGGAA-3′ and the like) and the like.

When evaluating the binding of Spi-B to the above-described DNA, the binding can be achieved using a method known per se, for example, binding assay, a method utilizing surface plasmon resonance (for example, use of Biacore (registered trademark)), gel shift assay and the like, using the isolated Spi-B polypeptide and the DNA having a GGA (A/T) core sequence. A fragment of the Spi-B polypeptide comprising a site capable of mediating the binding action (Ets domain and the like) may be used.

In another aspect, functions of Spi-B include binding to IRF-7.

When evaluating the binding of Spi-B to IRF-7, the binding can be achieved using a method known per se, for example, binding assay, a method utilizing surface plasmon resonance (for example, use of Biacore (registered trademark)), yeast two-hybrid assay and the like, using the isolated Spi-B polypeptide and the isolated IRF-7 polypeptide.

A substance judged as a result of the comparison to inhibit the function of Spi-B can be selected as a substance capable of inhibiting type I IFN production (or a substance capable of inhibiting the expression of a type I IFN gene).

As stated above, the expression of Spi-B is high in dendritic cells, particularly in pDC; therefore, a substance obtained by the screening method of the present invention is advantageous in inhibiting type I IFN production in dendritic cells, particularly in pDC. Because pDC possesses a potent capability of IFN-α production, a substance obtained by the screening method of the present invention is particularly advantageous in inhibiting IFN-α production in pDC.

Furthermore, as stated above, because Spi-B promotes type I IFN production in cooperation with IRF-7, and also because IRF-7 is profoundly involved in type I IFN production via TLR7 or 9, a substance obtained by the screening method of the present invention is advantageous in inhibiting type I IFN production due to stimulation via TLR7 or 9.

A substance obtained by the screening method of the present invention, like the above-described antisense nucleic acid or siRNA against Spi-B and the like, is useful as a candidate substance for a prophylactic agent/inhibitor for a disease associated with excess production of type I IFN.

### 3. Type I IFN Production Inducer

The present invention provides a type I IFN production inducer comprising a vector capable of expressing Spi-B and an expression vector capable of expressing IRF-7 in combination.

Because Spi-B induces type I IFN production in cooperation with IRF-7, it is possible to potently induce type I IFN production by administering an expression vector capable of expressing Spi-B and a vector capable of expressing IRF-7 in combination. Because type I IFNs possess antiviral action and antitumor action, the type I IFN production inducer of the present invention is useful as a prophylactic/therapeutic agent for viral infections and tumors.

Spi-B is defined as described in the (1. Type I IFN production inhibitor) section.

IRF-7 is a publicly known transcription factor belonging to the interferon control transcription factor family. The IRF-7 used in the present invention is derived from a mammal. Examples of the mammal include, but are not limited to, laboratory animals such as mice, rats, hamsters, guinea pigs, and other rodents, and rabbits; domestic animals such as swines, bovines, goat, horses, sheep, and minks; companion animals such as dogs and cats; and primates such as humans, monkeys, cynomolgus monkeys, rhesus monkeys, marmosets, orangutans, and chimpanzees. Representative nucleotide sequences and amino acid sequences of human and mouse IRF-7 are registered with the GenBank as follows:

### [Human IRF-7]

Nucleotide sequences (cDNA sequences): Accession numbers NM_{—}004029 (version NM_{—}004029.2) (SEQ ID NO:7), NM_{—}001572 (version NM_{—}001572.3) (SEQ ID NO:9), and NM_{—}004031 (version NM_{—}004031.2) (SEQ ID NO:11)
Amino acid sequences: Accession numbers NP_{—}004020 (version NP_{—}004020.1) (SEQ ID NO:8), NP_{—}001563 (version NP_{—}001563.2) (SEQ ID NO:10), and NP_{—}004022 (version NP_{—}004022.2) (SEQ ID NO:12)

### [Mouse IRF-7]

Nucleotide sequence (cDNA sequences): Accession number NM_{—}016850 (version NM_{—}016850.2) (SEQ ID NO:13)
Amino acid sequence: Accession number NP_{—}058546 (version NP_{—}058546.1) (SEQ ID NO:14)

In the vector capable of expressing Spi-B or IRF-7, nucleic acids (preferably DNA) that encode these polypeptides are operably linked to a promoter capable of exhibiting promoter activity in the cells of a recipient mammal (preferably human or mouse). The vector is capable of expressing the Spi-B or IRF-7 polypeptide under the control of the promoter.

The promoter used is not particularly limited, as far as it is capable of functioning in the cells of the recipient mammal. Useful promoters include poll-system promoters, polII-system promoters, polIII-system promoters and the like. Specifically, viral promoters such as the SV40-derived initial promoter and cytomegalovirus LTR, mammalian constitutive protein gene promoters such as the β-actin gene promoter, RNA promoters such as the tRNA promoter, and the like are used.

The vector capable of expressing Spi-B or IRF-7 preferably contains a transcription termination signal, i.e., a terminator region, downstream of the nucleic acid that encodes Spi-B or IRF-7. Furthermore, the vector may further contain a selection marker gene for transformed cell selection (a gene that confers resistance to a drug such as tetracycline, ampicillin, or kanamycin, a gene that compensates for auxotrophic mutations, and the like).

Although the choice of vector used in the expression is vector is not particularly limited, suitable vectors for administration to mammals such as humans include viral vectors such as retroviruses, adenoviruses, and adeno-associated viruses. Adenoviruses, in particular, have advantages such as very high gene transfer efficiency and transferability to non-dividing cells. Because the integration of transgenes into host chromosome is extremely rare, however, the gene expression is transient and normally persists only for about 4 weeks. In view of the persistence of therapeutic effect, it is also preferable to use an adeno-associated virus, which offers a relatively high gene transfer efficiency, which can be transferred to non-dividing cells as well, and which can be integrated into chromosome via an inverted terminal repeat (ITR).

When using in combination a vector capable of expressing Spi-B (hereinafter referred to as the Spi-B vector) and a vector capable of expressing IRF-7 (hereinafter referred to as the IRF-7 vector), the dosing times of the Spi-B vector and the IRF-7 vector are not limited; the Spi-B vector and the IRF-7 vector may be administered to the recipient simultaneously, or administered at a time lag. The doses of the Spi-B vector and the IRF-7 vector are not particularly limited, as far as prophylaxis/treatment of the indicated disease can be accomplished, and the doses can be chosen as appropriate according to the recipient, the route of administration, disease, combination and the like.

The mode of administration of the Spi-B vector and the IRF-7 vector is not particularly limited, as far as the Spi-B vector and the IRF-7 vector are combined at the time of administration. Examples of such modes of administration include (1) administration of a single preparation obtained by simultaneously preparing the Spi-B vector and the IRF-7 vector, (2) simultaneous administration via the same route of administration of two different preparations obtained by preparing the Spi-B vector and the IRF-7 vector as separate preparations, (3) administration via the same route of administration of two different preparations obtained by preparing the Spi-B vector and the IRF-7 vector as separate preparations, at a time lag, (4) simultaneous administration via different routes of administration of two different preparations obtained by preparing the Spi-B vector and the IRF-7 vector as separate preparations, (5) administration via different routes of administration of two different preparations obtained by preparing the Spi-B vector and the IRF-7 vector as separate preparations, at a time lag (for example, administration in the order of Spi-B vector→IRF-7 vector, or administration in the reverse order) and the like.

The type I IFN production inducer of the present invention can be prepared by blending the Spi-B vector and/or the IRF-7 vector and a pharmaceutically acceptable carrier using a conventional method.

Examples of the pharmaceutically acceptable carrier include, but are not limited to, excipients such as sucrose and starch; binders such as cellulose and methylcellulose; disintegrants such as starch and carboxymethylcellulose; lubricants such as magnesium stearate and Aerosil; flavoring agents such as citric acid and menthol; preservatives such as sodium benzoate and sodium hydrogen sulfite; stabilizers such as citric acid and sodium citrate; suspending agents such as methylcellulose and polyvinylpyrrolidone; dispersing agents such as surfactants; diluents such as water and physiological saline; base waxes; and the like.

To promote the introduction of a polynucleotide into a cell, the inducer of the present invention can further comprise a reagent for nucleic acid introduction. When the polynucleotide is incorporated in a viral vector, particularly in a retroviral vector, retronectin, fibronectin, polybrene or the like can be used as a reagent for gene transfer. When the polynucleotide is incorporated in a plasmid vector, a cationic lipid such as lipofectin, lipfectamine, DOGS (transfectam), DOPE, DOTAP, DDAB, DHDEAB, HDEAB, polybrene, or poly(ethyleneimine) (PEI) can be used.

Preparations suitable for oral administration include liquids, capsules, sachets, tablets, suspensions, emulsions and the like.

Preparations suitable for parenteral administration (for example, subcutaneous injection, intramuscular injection, topical injection, intraperitoneal administration and the like) include aqueous and non-aqueous isotonic sterile injectable liquids, which may contain an antioxidant, a buffer solution, a bacteriostatic agent, an isotonizing agent and the like. Aqueous and non-aqueous sterile suspensions can also be mentioned, which may contain a suspending agent, a solubilizer, a thickening agent, a stabilizer, an antiseptic and the like. These preparations can be encapsulated in containers such as ampoules and vials for unit dosage or a plurality of dosages. It is also possible to freeze-dry the active ingredient and a pharmaceutically acceptable carrier, and store the preparation in a state that may be dissolved or suspended in an appropriate sterile vehicle just before use.

When the Spi-B vector and the IRF-7 vector are simultaneously prepared and used as a single preparation, the content amount of the Spi-B vector in the pharmaceutical of the present invention varies depending on the form of the preparation, and is normally about 0.1% to 99.9% by weight, preferably about 1% to 99% by weight, more preferably about 10% to 90% by weight, relative to the entire preparation.

The content amount of the IRF-7 vector in the pharmaceutical of the present invention varies depending on the form of the preparation, and is normally about 0.1% to 99.9% by weight, preferably about 1% to 99% by weight, more preferably about 10% to 90% by weight, relative to the entire preparation.

In the pharmaceutical of the present invention, the content amount of ingredients other than the Spi-B vector and the IRF-7 vector varies depending on the form of the preparation, and is normally about 0.2% to 99.8% by weight, preferably about 2% to 98% by weight, preferably about 20% to 90% by weight, relative to the entire preparation.

A blending ratio of the above-described Spi-B vector and IRF-7 vector in the inducer of the present invention can be chosen as appropriate according to the recipient, the route of administration, disease and the like.

Because the preparation thus obtained is safe and of low toxicity, it can be administered to, for example, humans and other warm-blooded animals (for example, rats, mice, hamsters, rabbits, sheep, goat, pigs, bovines, horses, cats, dogs, monkeys, chimpanzees, birds and the like).

The dose of the Spi-B vector varies depending on the route of administration, target disease, symptoms, patient's age and the like; generally speaking, in the case of parenteral administration, it is advantageous that the dose be about 0.001 to about 500 mg/kg per day in a patient (assuming a 60 kg body weight).

The dose of the IRF-7 vector varies depending on the route of administration, target disease, symptoms, patient's age and the like; generally speaking, in the case of parenteral administration, it is advantageous that the dose be about 0.001 to about 500 mg/kg per day in a patient (assuming a 60 kg body weight).

When the Spi-B vector and the IRF-7 vector are prepared as separate preparations, the content amounts may be the same as those shown above.

When the above-described Spi-B vector and IRF-7 vector are prepared as separate preparations and administered in combination, the preparation containing the Spi-B vector and the preparation containing the IRF-7 vector may be administered simultaneously; however, the preparation containing the IRF-7 vector may be administered in advance, after which the preparation containing the Spi-B vector may be administered, and the preparation containing the Spi-B vector may be administered in advance, after which the preparation containing the IRF-7 vector may be administered. When the two preparations are administered at a time lag, the time lag varies depending on the active ingredient administered, dosage form, and the method of administration; for example, when the preparation containing the IRF-7 vector is administered in advance, a method is available wherein the preparation containing the Spi-B vector is administered within 1 minute to 3 days, preferably within 10 minutes to 1 day, more preferably within 15 minutes to 1 hour, after administration of the preparation containing the IRF-7 vector. When the preparation containing the Spi-B vector is administered in advance, a method is available wherein the preparation containing the IRF-7 vector is administered within 1 minute to 1 day, preferably within 10 minutes to 6 hours, more preferably within 15 minutes to 1 hour, after administration of the preparation containing the Spi-B vector.

The inducer of the present invention is very useful not only for the above-described in vivo use applications, but also as a reagent for research concerning type I IFN production in vitro.

The present invention is hereinafter described in more detail by means of the following Examples, to which, however, the invention is not limited in any way.

### EXAMPLES

### Example 1

### Materials and Methods

### Plasmids

The vector for luciferase expression driven by the IFN-α4 promoter was generated by subcloning the promoter region of the mouse IFN-α4 gene into the pGL3 vector (Promega) (non-patent document 9). The IFN-α4 promoter region was amplified by PCR using the primers shown below.

(SEQ ID NO: 16)

| | |
|---|---|
| Sense primer; | 5′-CCCCCACACTTTACTTTTTTGACAGAA-3′ |

(SEQ ID NO: 17)

| | |
|---|---|
| Antisense primer; | 5′-TACAGGTTCTCTGAGAGCCTGCTGTGT-3′ |

The mouse IFN-α4 promoter used was a region consisting of the 433 bp from −486 bp to −54 bp upstream of the transcription initiation site of the IFN-α4 gene. At −163 to −152 of the region, a positive regulatory domain-like element (PRD-LE) has been identified as a site important to the gene expression (E. C. Zwarthoff, et al., Nucleic Acid Research 13:791-804, 1985; K. Honda et al., Int Immunol 17:1367-1378, 2005). In this mouse IFN-α4 promoter, the 135 bp from −188 to −54, including PRD-LE, is highly homologous to the human IFN-α4% promoter (72.2%).

The plasmid for luciferase expression driven by the IFN-β promoter was generated by subcloning the promoter region of the mouse IFN-β gene into the pGL3-Basic vector. The IFN-β promoter region was amplified by PCR using the primers shown below.

(SEQ ID NO: 18)

| | |
|---|---|
| Sense primer; | 5′-AGCTTGAATAAAATGAATATTAGAAGC-3′ |

(SEQ ID NO: 19)

| | |
|---|---|
| Antisense primer; | 5′-CAAGATGAGGCAAAGGCTGTCAAAGGC-3′ |

The mouse IFN-β promoter used was a region comprising −140 by to +42 by upstream of the transcription initiation site of the IFN-β gene. At −98 to −52 in the region, four positive regulatory domains (PRD) (PRDI, PRDII, PRDIII, PRDIV) have been identified as sites that are important to the gene expression (K. Honda et al., Int Immunol 17:1367-1378, 2005). This mouse IFN-β promoter is highly homologous to −137 to +41 upstream of the transcription initiation site of human IFN-β (79%). Contained in this region are all of PRDI, PRDII, PRDIII, and PRDIV.

Expression vectors for mouse Spi-B, IRF-1, IRF-3, IRF-5 and IRF-7 were generated as described below. An HA-tagged mouse Spi-B cDNA fragment was amplified by PCR from an Spi-B cDNA clone (msh30167) as the template and subcloned into CSII-EF-MCS-IRES2-venus (CSII-EF-HA-mSpiB-IRES2-venus). For siRNA experiments, CSII-EF-HA-mSpiB subcloned into CSII-EF-MCS was used. A FLAG-tagged mouse IRF-1 cDNA fragment was amplified by PCR from an IRF-1 dDNA clone (msj01193) as the template and subcloned into pEF-BOS (pEF-BOS-FLAG-mIRF-1). A FLAG-tagged mouse IRF-3 cDNA fragment was amplified by PCR from an IRF-3 cDNA clone (3110001G18) and subcloned into pEF-BOS (pEF-BOS-FLAG-mIRF-3). A FLAG-tagged mouse IRF-5 cDNA fragment was amplified by PCR from an IRF-5 cDNA clone (F830012G18) and subcloned into pEF-BOS (pEF-BOS-FLAG-mIRF-5). A FLAG-tagged mouse IRF-7 cDNA fragment was amplified by PCR from a CpG DNA-stimulated GM-CSF BMDC cDNA library and subcloned into pEF-BOS (pEF-BOS-FLAG-mIRF-7). A FLAG-tagged mouse IRF-8 cDNA fragment was amplified from an IRF-8 cDNA clone (9830117K07) by PCR and subcloned into pEF-BOS (pEF-BOS-FLAG-mIRF-8).

### Luciferase Assay

293T cells were seeded on 24-well plates (7×10⁴ cells/well) and cultured overnight. These cells are transiently transfected with luciferase reporter plasmid (60 ng) together with indicated amounts of expression plasmids using Lipofectamine 2000 (Invitrogen). Cell lysates were prepared 24 h after transfection and luciferase activity was measured by Dual-luciferase reporter assay system (Promega).

Effects of Mouse Spi-B siRNA

293T cells were seeded on 24-well plates (1.7×10⁵ cells/well) and cultured overnight. These cells are transiently transfected with luciferase reporter plasmid (70 ng) together with indicated amounts of expression plasmids and siRNA using Lipofectamine 2000 (Invitrogen). Cell lysates were prepared 24 h after transfection and luciferase activity was measured. Total RNA was also prepared from each well, and expression level of Spi-B was analyzed by quantitative PCR.

The siRNA against mouse Spi-B used was a mixture of the four different siRNAs shown below.

siRNA-1

| | | |
|---|---|---|
| Sense: | AGACAGGCGAAAUCCGCAAUU | (SEQ ID NO: 20) |
| Antisense: | UUGCGGAUUUCGCCUGUCUUU | (SEQ ID NO: 21) |
| siRNA-2 | | |
| Sense: | UGUCUGAGCACUCCGCUAAUU | (SEQ ID NO: 22) |
| Antisense: | UUAGCGGAGUGCUCAGACAUU | (SEQ ID NO: 23) |
| siRNA-3 | | |
| Sense: | GCGCAUGACGUAUCAGAAGUU | (SEQ ID NO: 24) |
| Antisense: | CUUCUGAUACGUCAUGCGCUU | (SEQ ID NO: 25) |
| siRNA-4 | | |
| Sense: | CGACCUGUAUGUUGUGUUUUU | (SEQ ID NO: 26) |
| Antisense: | AAACACAACAUACAGGUCGUU | (SEQ ID NO: 27) |

siRNA-1 and 3 target the coding region of Spi-B mRNA, whereas siRNA-2 and 4 target the non-coding region.

### [Results]

### Spi-B Expression in DC Subsets

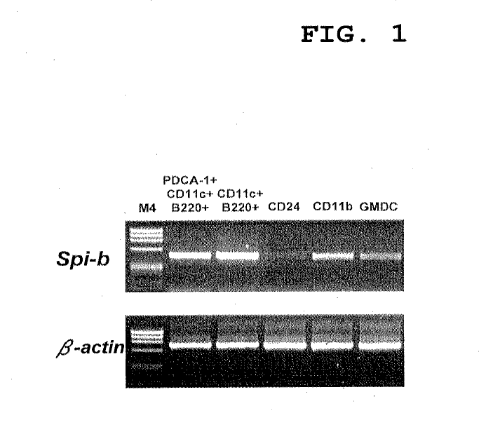

The present inventors have first analyzed Spi-B gene expression in various types of DCs by RT-PCR. Bone marrow (BM) cells can give rise to both pDC and cDC when cultured in the presence of Flt3L (Gilliet, M. et al., J Exp Med, 195, 953-8 (2002)). pDC and cDC can be defined as CD11c⁺B220⁺ and CD11c⁺B220⁻ cells, respectively. CD11c⁺B220⁻ cDC can be further divided into CD24^{high}CD11b^{low} cDC and CD24^{low}CD11b^{high} cDC (Naik, S. H. et al., J Immunol 174, 6592-7 (2005)). When cultured with GM-CSF, BM cells can also give rise to cDC, but not to pDC. GM-CSF-induced cDC is different from Flt3L-induced cDC in terms of function and gene expression patterns. The present inventors have first compared gene expression profiles among these four types of DCs through the DNA microarray analysis based on the gcRMA method and found that Spi-B expression was highest in pDC (pDC:15207.0, CD24:353.4, CD11b cDC:4447.0, GM-CSF-induced cDC:969.8). High expression of Spi-B in pDC was confirmed by RT-PCR (FIG. 1). Because PDCA-1 is specifically expressed in pDC (Blasius, A. L. et al., J Immunol 177, 3260-5 (2006)), the present inventors have also tested Spi-B expression in the CD11c⁺B220⁺ PDCA-1⁺ population. Spi-B expression was observed also in this population (FIG. 1). From these results, it was shown that Spi-B is abundantly expressed in pDC.

### Effects of Spi-B Expression on Type I IFN Promoters

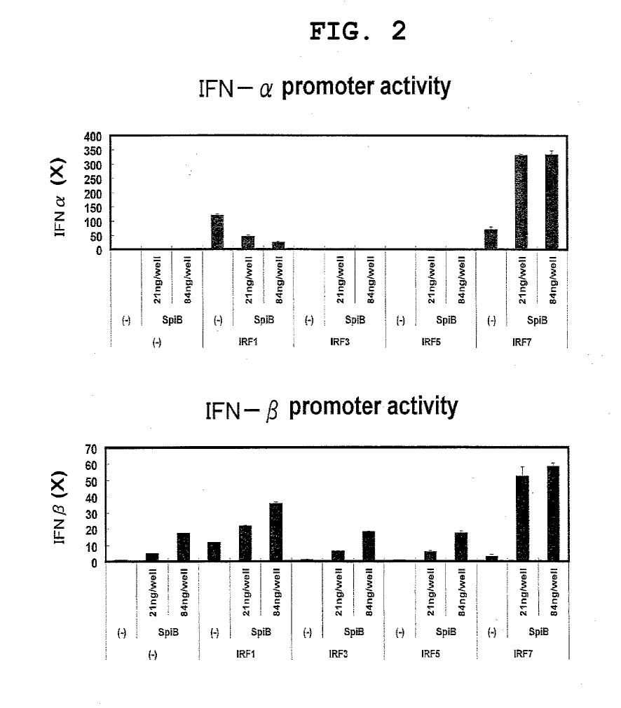

Spi-B belongs to the Ets transcription factor family (non-patent documents 12 and 13). The family members can transactivate the enhancers or promoters of target genes coordinately with IRF family members. IRF-7 is critical for pDC to produce type I IFNs including IFN-α and IFN-β (non-patent document 7). The present inventors have investigated whether Spi-B can transactivate the type I IFN promoters. For this purpose, the present inventors have performed the luciferase assay (FIG. 2). Expression of IRF-7 activated the IFN-α promoter (FIG. 2A). Although expression of Spi-B alone failed to activate the IFN-α promoter, its expression significantly upregulated IRF-7-induced transactivation. Meanwhile, although IRF-1 expression alone could transactivate the promoter, coexpression of Spi-B rather suppressed IRF-1-induced transactivation.

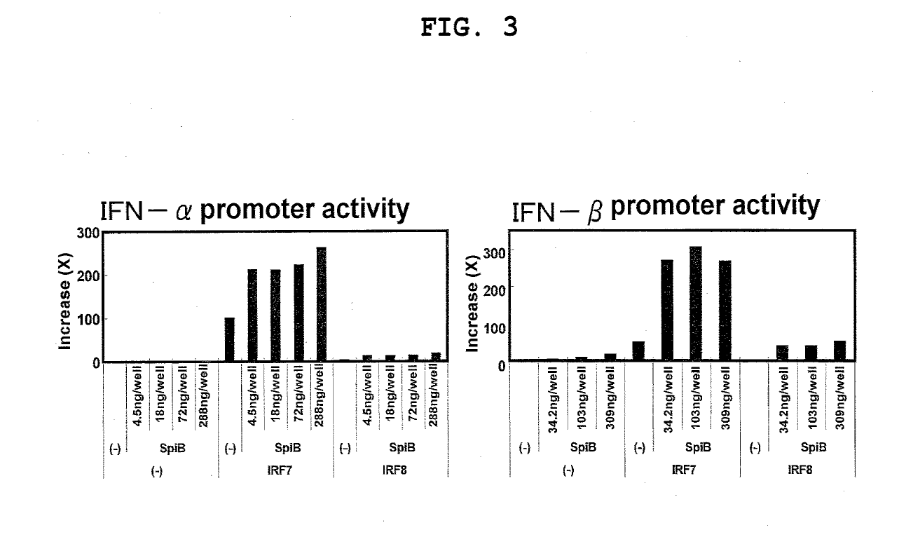

The present inventors have also tested the effects on the IFN-β promoter (FIG. 2B). Spi-B expression alone enhanced the promoter activity. IRF-7 could only marginally activate the promoter. Notably, Spi-B and IRF-7 synergistically enhanced transactivation of the IFN-β promoter. Coexpression of neither IRF-3 nor IRF-5 with Spi-B upregulated the promoter activation. IRF-1 enhanced Spi-B-induced transactivation, but this effect is additive, given that IRF-1 expression alone can transactivate the IFN-β promoter. IRF-8, with Spi-B, exhibited a slight synergistic activation on type I IFN promoters, which activation, however, was much weaker than the synergistic effect of IRF-7 and Spi-B (FIG. 3).

Mouse Spi-B siRNA can Suppress Mouse Spi-B-Induced Transactivation.

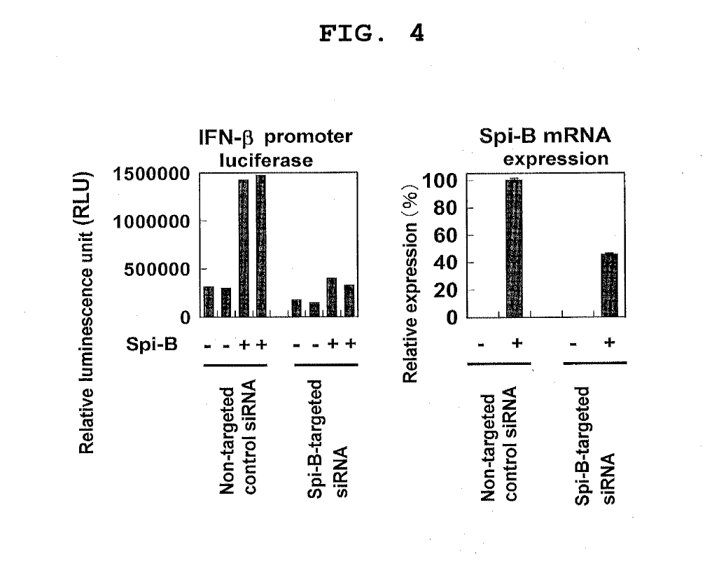

The present inventors have next tested the effects of mouse Spi-B siRNA. In the presence of control siRNA which does not target Spi-B, Spi-B-induced transactivation of the IFN-β promoter was observed (FIG. 4A). However, Spi-B-induced transactivation was abrogated in the presence of mouse Spi-B-targeted siRNA. In mouse Spi-B-targeted siRNA transfected cells, mouse Spi-B mRNA expression level was decreased to 40% of control siRNA transfected cells.

### pDC is Generated in Spi-B-Deficient Mice.

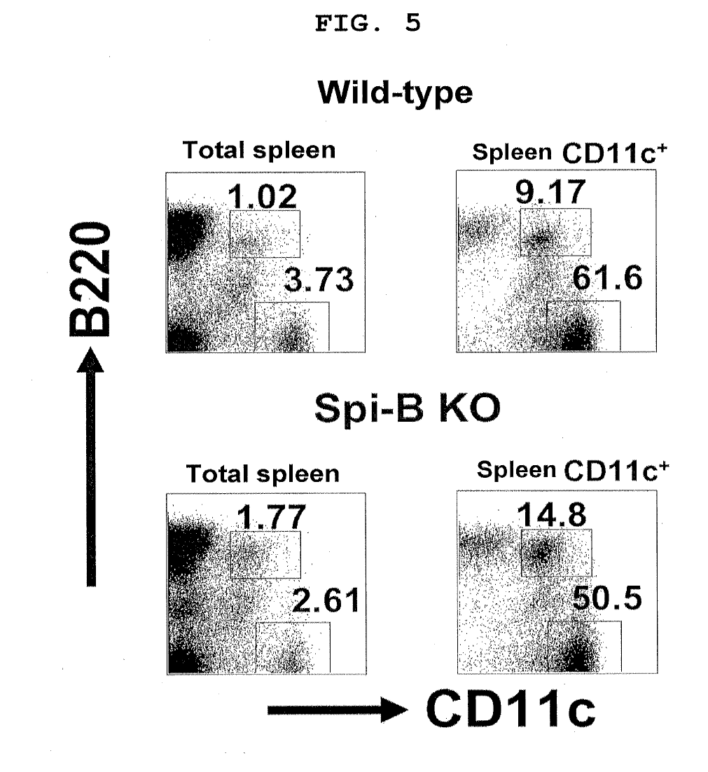

In order to elucidate in vivo roles of Spi-B, the present inventors have generated Spi-B-deficient mice. The mutant mice are born healthy without gross abnormality as described previously (Su, G. H. et al., Embo J 16, 7118-29 (1997)). In the spleen, CD11c⁺B220⁺ and CD11c⁺B220⁻ cell populations were detected in comparable percentages between wild-type mice and Spi-B-deficient mice (FIG. 5). pDC was detected also in the BM (non-patent document 4). In Spi-B-deficient mice, CD11c⁺B220⁺ cells were decreased to about 50% of wild-type mice. Thus, it was shown that Spi-B is dispensable for pDC generation.

pDC defects in Spi-B-Deficient Mice

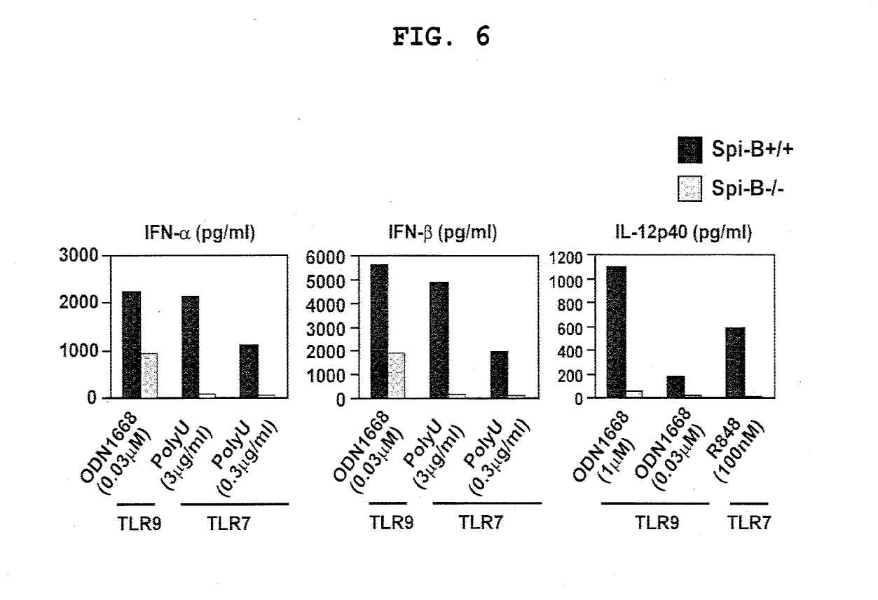

The present inventors have prepared pDCs from wild-type mice and Spi-B-deficient mice and analyzed cytokine produced from pDCs when stimulated with various kinds of TLR7 and TLR9 agonists (FIG. 6). Wild-type pDC produced significant amounts of IFN-α, IFN-β and IL-12p40 in response to those TLR agonists. The cytokine production was severely impaired in Spi-B-deficient pDC. The results suggest that Spi-B is required for in vitro pDC responses to TLR7 and TLR9.

Serum Cytokine Levels upon TLR7 Agonist Injection

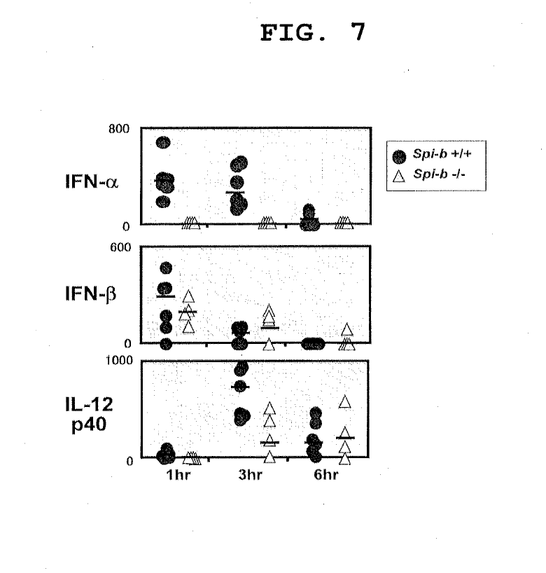

A TLR7 agonist, polyU RNA, increases serum cytokine levels when injected into wild-type mice. This reaction is already known to be dependent on TLR7. Wild-type mice showed elevation of serum IFN-α, IFN-β and IL-12p40 levels after intravenous injection of polyU (FIG. 7). The elevation was impaired in Spi-B-deficient mice. The impairment was prominent in serum IFN-α levels. Among these three cytokines, production of IFN-α depends solely on pDC, while production of the other cytokines depends on pDC and cDC. The results suggest that Spi-B is required for in vivo pDC responses to TLR7 agonists.

### Example 2

As in Example 1, the effect of human Spi-B expression vector on the expression of luciferase driven by a mouse IFN-β promoter, and the effect of human Spi-B siRNAs thereon were examined by luciferase assay.

### [Materials and Methods]

The plasmid used for the expression of luciferase driven by the mouse IFN-β promoter was the same as that used in Example 1.

Human Spi-B expression vectors were prepared as described below. An HA-tagged human Spi-B cDNA fragment was amplified from the template Spi-B cDNA (Open Biosystems 4309499) by PCR, and subcloned into CSII-EF-MCS to obtain CSII-EF-HA-hSpiB, which was used.

The luciferase assay and a confirmatory test for the effect of Spi-B siRNA were performed in the same manner as Example 1.

The siRNAs against human Spi-B and control siRNA used had the sequences shown below.

siRNA-1

| | | |
|---|---|---|
| Sense: | GAACUUCGCUAGCCAGACCUU | (SEQ ID NO: 28) |
| Antisense: | GGUCUGGCUAGCGAAGUUCUU | (SEQ ID NO: 29) |
| siRNA-2 | | |
| Sense: | CUGGACAGCUGCAAGCAUUUU | (SEQ ID NO: 30) |
| Antisense: | AAUGCUUGCAGCUGUCCAGUU | (SEQ ID NO: 31) |
| siRNA-3 | | |
| Sense: | CAGAUGGCGUCUUCUAUGAUU | (SEQ ID NO: 32) |
| Antisense: | UCAUAGAAGACGCCAUCUGUU | (SEQ ID NO: 33) |
| siRNA-4 | | |
| Sense: | GAGGAAGACUUACCGUUGGUU | (SEQ ID NO: 34) |
| Antisense: | CCAACGGUAAGUCUUCCUCUU | (SEQ ID NO: 35) |

The control siRNA used was ON-TARGETplus Non-targeting Pool (Dharmacon D-001810-10).

### [Results]

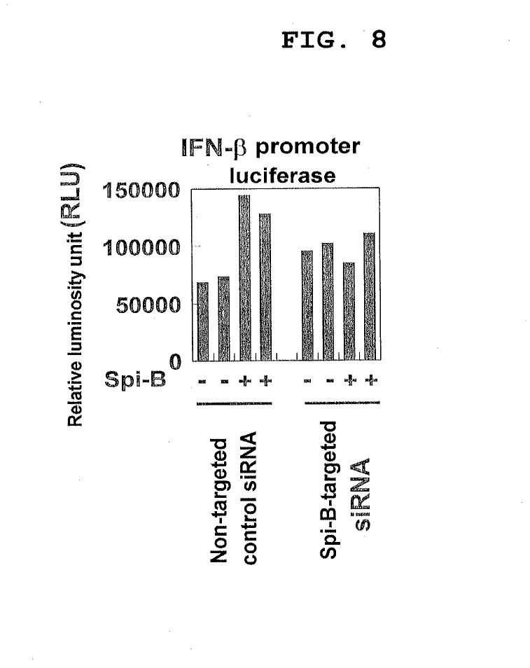

As with mouse Spi-B, in the presence of the control siRNA, which did not target Spi-B, transactivation of the human IFN-β promoter was induced by human Spi-B. However, in the presence of an siRNA that targeted human Spi-B, the transactivation induced by human Spi-B was suppressed (FIG. 8).

### Example 3

To clarify the molecular mechanism by which Spi-B and IRF-7 cooperatively activate a type I IFN promoter, an examination was made to determine whether Spi-B and IRF-7 associated with each other.

### [Materials and Methods]

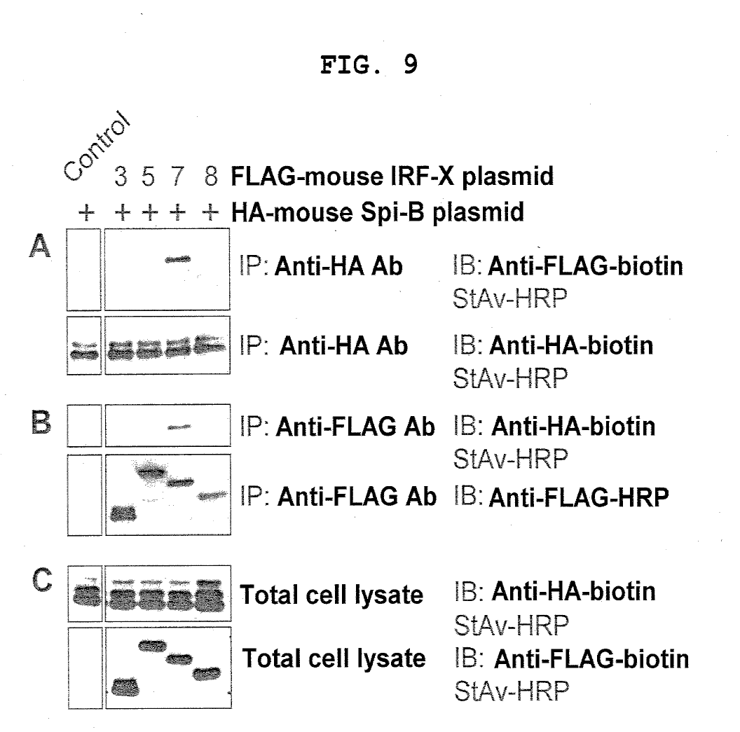

293T cells were seeded to a 6 cm dish (1.4×10⁶ cells/dish) and cultured overnight. Using lipofectamine 2000 (Invitrogen), a plasmid that encodes the HA-tagged mouse Spi-B gene (HA-SpiB-IRES2-venus, 4 μg) or a plasmid that encodes each FLAG-tagged mouse IRF family gene (pEF-BOS-FLAG-mIRF-3, pEF-BOS-FLAG-mIRF-5, pEF-BOS-FLAG-mIRF-7, pEF-BOS-FLAG-mIRF-8, 4 μg each) was transiently transfected to the 293 cells. As the control plasmid for pEF-BOS-FLAG-mIRF 3, 5, 7, and 8, pEF-BOS was used. 24 hours after the transfection, a cell extract was prepared using a RIPA buffer (50 mM Tris-HCl, 150 mM NaCl, 1% (v/v) NP-40, 0.5% (w/v) DOC, 0.1% (w/v) SDS, pH 8.0), and immunoprecipitated with an anti-HA antibody (MBL 561) or anti-FLAG antibody (SIGMA F1804); the immunoprecipitate was subjected to SDS-PAGE, and then transferred to a PVDF membrane (FIGS. 9A, B). Separately, the cell extract was directly subjected to SDS-PAGE, without performing immunoprecipitation, and then transferred to a PVDF membrane (FIG. 9C). Furthermore, immunoblotting was performed using a biotinylated anti-HA antibody (Roche 2158167) or a biotinylated anti-FLAG antibody (M2, SIGMA F9291) as the primary antibody. To detect the primary antibody, Horseradish Peroxidase (HRP)-labeled streptavidin (GE Healthcare RPN1231) was used. When the primary antibody was not used (FIG. 9B), an HRP-labeled anti-FLAG antibody (M2, SIGMA A8592) was used. Subsequently, a chemiluminescent substrate (PerkinElmer NEL103001EA) was reacted, and a band was detected by sensing chemiluminescence due to the HRP using an X-ray film.

### [Results]

The 293 cells were allowed to express Spi-B or IRF family members, and the cell extract was analyzed (FIG. 9). In the Spi-B immunoprecipitate, IRF-7 was detected, but none of IRF-3, 5, and 8 was detected (FIG. 9A). Meanwhile, in the IRF-7 immunoprecipitate, Spi-B was detected, but Spi-B was not detected in any of the IRF-3, 5, and 8 immunoprecipitates (FIG. 9B). These results showed that Spi-B was strongly associated with IRF-7. This association was stronger than the association with any other IRF family member; the intensity of association is thought to be contributory to the activation of type I IFN promoter.

### Example 4

pDC expresses various membrane proteins with maturity and differentiation. Ly49Q is a membrane protein highly expressed in dendritic cells, particularly in pDC, and its expression is enhanced with the maturation of pDC (Toyama-Sorimachi, N., Y. Omatsu, A. Onoda, Y. Tsujimura, T. Iyoda, A. Kikuchi-Maki, H. Sorimachi, T. Dohi, S. Taki, K. Inaba, and H. Karasuyama. 2005. Inhibitory NK receptor Ly49Q is expressed on subsets of dendritic cells in a cellular maturation- and cytokine stimulation-dependent manner. J. Immunol. 174:4621-4629. Omatsu, Y., T. Iyoda, Y. Kimura, A. Maki, M. Ishimori, N. Toyama-Sorimachi, and K. Inaba. 2005. Development of murine plasmacytoid dendritic cells defined by increased expression of an inhibitory NK receptor, Ly49Q. J. Immunol. 174:6657-6662). Analysis of Ly49Q-deficient mice has shown that Ly49Q plays an important role in the production of cytokines, including type I IFNs, from pDC stimulated with TLR7 and TLR9 (L.-H. Tai, M.-L. Goulet, S. Belanger, N. Toyama-Sorimachi, N. Fodil-Cornu, S. M. Vidal, A. D. Troke, D. W. McVicar, A. P. Makrigiannis. 2008. Positive regulation of plasmacytoid dendritic cell function via Ly49Q recognition of class I MHC. J. Exp. Med. 205:3187-3199.). With this in mind, an investigation was performed to determine whether Spi-B is involved in the expression of the Ly49Q gene.

### [Materials and Methods]

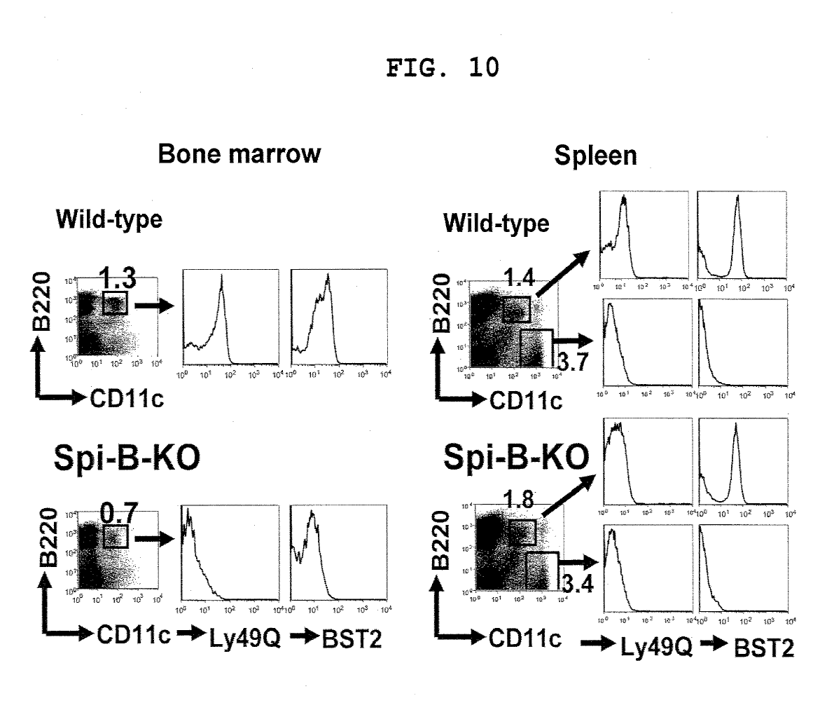

Myelocytes and splenocytes were prepared from a wild-type mouse and an Spi-B-deficient mouse, stained with the combination of a Fluorescein isothiocyanate (FITC)-labeled anti-Ly49Q antibody (MBL D160-4), a phycoerythrin (PE)-labeled anti-B220 antibody (RA3-6B2, ebioscience 12-0452-85), a Biotin-labeled anti-CD11c antibody (N418, ebioscience 13-0112-82), and Cychrome(CyC)-labeled streptavidin, or with the combination of an FITC-labeled anti-CD11c antibody (N418, ebioscience 11-0114-82), a PE-labeled anti-B220 antibody (RA3-6B2, ebioscience 12-0452-85), a Biotin-labeled anti-bone marrow stromal cell antigen 2 (BST2) antibody (PDCA-1, Miltenyi Biotec 130-091-964), and CyC-labeled streptavidin, and analyzed by flow cytometry (FACS Caliber) (FIG. 10).

Myelocytes were collected from a wild-type mouse and an Spi-B-deficient mouse; using an FITC-labeled anti-BST2 antibody (PDCA-1, Miltenyi Biotec 130-091-961), a PE-labeled anti-B220 antibody (RA3-6B2, ebioscience 12-0452-85), and an allophycocyanin (APC)-labeled anti-CD11c antibody (N418, ebioscience 17-0114-82), CD11c-positive B220-positive BST2-positive cells were collected by sorting (FACS Vantage); RNA was prepared; and gene expression analysis was performed using a DNA microarray (Affymetrix Mouse Genome 430 2.0Array).

A portion from downstream of the 3′ of a region estimated to be the first exon of the Ly49Q gene to upstream of the 5′ of the start of Exon 1 (full length 3698 bp) was amplified using the two different primers:

090109Ly49Qpro-F2:
(SEQ ID NO: 36)
5′-CTAGCCCGGGCTCGAGCCTTCAAAGTAGAACTGAAGCATTC-3′
090107Ly49Qpro-R3:
(SEQ ID NO: 37)
5′-CCGGAATGCCAAGCTTTTCTGCATCAATCCTGATCTCATGTC-3′

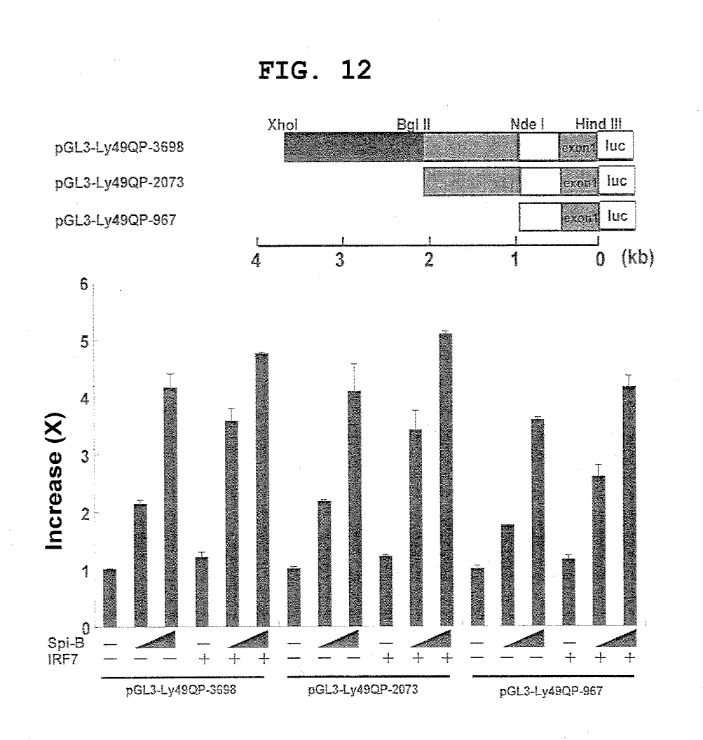

with the DNA of the ES cell line Bruce4 as the template, and subcloned into the XhoI-HindIII site upstream of the 5′ of the luciferase gene in a plasmid (pGL3-Basic vector, Promega E-1751), whereby pGL3-Ly49QP-3698 was prepared (FIG. 12). Also, by cleaving pGL3-Ly49QP-3698 with XhoI and BglII, blunting the cut ends, and re-joining the cut ends, pGL3-Ly49QP-2073 was generated; by cleaving the same with XhoI and NdeI, blunting the cut ends, and re-joining the cut ends, pGL3-Ly49QP-967 was generated (FIG. 12). Furthermore, using the primer pair:

(SEQ ID NO: 38)
5′-CTAGCCCGGGCTCGAGacacttagctgcaattagcataac-3′
and
090107Ly49Qpro-R3,
or
(SEQ ID NO: 39)
5′-CTAGCCCGGGCTCGAGcttttcgatttggtcaaggaggag-3′
and
090107Ly49Qpro-R3

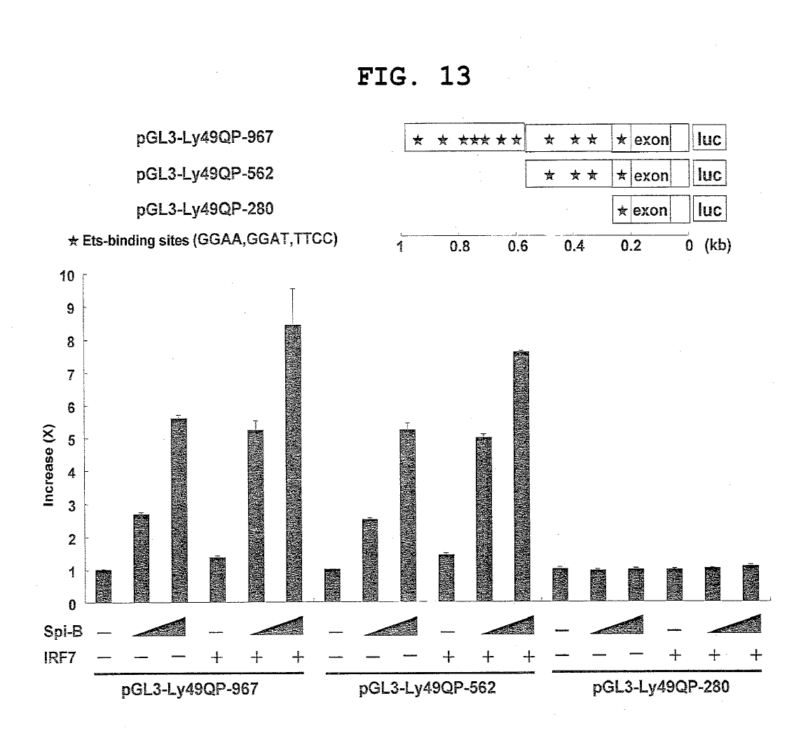

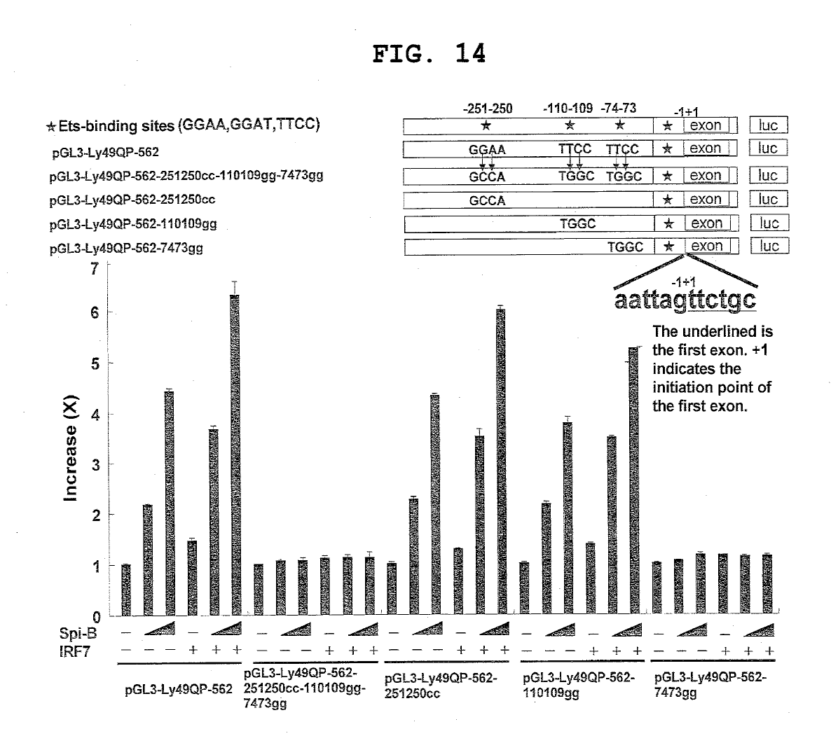

with the plasmid pGL3-Ly49QP-3698 as the template, each DNA fragment was amplified; and the fragment was inserted into the pGL3-Basic vector, whereby pGL3-Ly49QP-562 and pGL3-Ly49QP-280 were prepared, respectively (FIG. 13). At three putative Ets-binding sites in pGL3-Ly49QP-562, a mutation was introduced using the primer pair 251250CC-S and 251250CC-AS, the primer to pair 110109GG-S and 110109GG-AS, and the primer pair 7473GG-S and 7473GG-AS, with Quick Change Multi Site-Directed Mutagenesis Kit (Stratagene) (FIG. 14).

251250CC-S:
(SEQ ID NO: 40)
5′-TTACAAACCTGGAGCTGAGCCACCTGAGCTGCACATTTTT-3′,
251250CC-AS:
(SEQ ID NO: 41)
5′-AAAAATGTGCAGCTCAGGTGGCTCAGCTCCAGGTTTGTAA-3′
110109GG-S:
(SEQ ID NO: 42)
5′-CTGGCACAATATGTTACTTCTTGGCTTTGCTTTCAGAGTCAGGT
TT-3′
110109GG-AS:
(SEQ ID NO: 43)
5′-AAACCTGACTCTGAAAGCAAAGCCAAGAAGTAACATATTGTGCC
AG-3′
7473GG-S:
(SEQ ID NO: 44)
5′-TTTCAGAGTCAGGTTTCATTAAGCAATTGGCTCTTTTCGATTTG
GTCAGG-3′
7473GG-AS:
(SEQ ID NO: 45)
5′-CTTGACCAAATCGAAAAGAGCCAATTGCTTAATGAAACCTGACT
CTGAAA-3′

These various plasmids were used as luciferase reporter plasmids. 293T cells were seeded to a 24-well plate (7×10⁴ cells/well) and cultured overnight. Using lipofectamine 2000, each luciferase reporter plasmid (70 ng/well), along with an, Spi-B or IRF family member expression plasmid, was transfected to the 293T cells. The Spi-B expression plasmid was used at 0, 0.84, or 8.4 ng/well, and the control plasmid CSII-EF-MCS was added at 8.4, 7.56, or 0 ng/well, respectively, to make the amount of plasmid per well constant. The IRF-7 family member expression plasmid was used at 0 or 8.4 ng/well, and the control plasmid pEF-BOS was added at 8.4 or 0 ng/well, respectively. 24 hours after the transfection, a cell lysate was prepared, and luciferase activity was measured using a double luciferase reporter assay system (Promega).

### [Results]

In the spleens of the wild-type mice, CD11c-positive B220-positive cells were detected, and the expression of Ly49Q and BST2 was noted (FIG. 10). Meanwhile, in CD11c-positive B220-negative cells, the expression of Ly49Q and BST2 was not noted. In the spleens of the Spi-B-deficient mice, CD11c-positive B220-positive cells were detected; however, in these cells, the expression of Ly49Q decreased remarkably, although the expression of BST2 was maintained. Likewise in the bone marrow, the expression of Ly49Q in CD11c-positive B220-positive cells decreased remarkably in the Spi-B-deficient mice (FIG. 10). In an analysis using DNA microarray, the expression of the Ly49Q gene in CD11c-positive B220-positive BST2-positive cells decreased to an about quarter level in the Spi-B-deficient mice (wild-type:Spi-B-deficient=5709.2:1352). These results suggested that Spi-B might be essential to the expression of Ly49Q at the mRNA level.

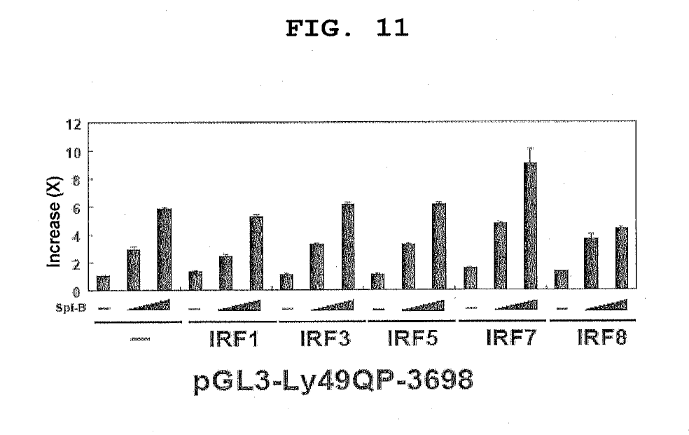

Furthermore, luciferase assay was performed to determine whether Spi-B directly activates a promoter of the Ly49Q gene. The 3698 bp DNA region of the Ly49Q gene, including the first exon, was activated by Spi-B, whose activation capacity was enhanced when it was co-expressed with IRF-7 (FIG. 11). Cooperative activation like this was not seen with other IRF family members, and this was a finding similar to the effect on type I IFN promoters. Next, various mutant plasmids lacking a DNA region were prepared and analyzed. When the region including the first exon was 562 bp, the activation by Spi-B, IRF-7 was maintained; however, when the region was deleted to 280 bp, the activation by Spi-B, IRF-7 disappeared (FIGS. 12 and 13). Furthermore, three sites to which an Ets family transcription factor was estimated to bind were present in the region essential to the activation by Spi-B, IRF-7; therefore, plasmids having all or any one of these sites mutated were generated and analyzed. As a result, of the three sites, the closest to the first exon (TTCC at −74,−73) was proven to be essential (FIG. 14).

### INDUSTRIAL APPLICABILITY

The type I IFN production inhibitor of the present invention is capable of potently inhibiting type I IFN production on the basis of the novel mechanism of suppression of Spi-B, and is useful as a prophylactic or therapeutic agent for various autoimmune diseases (for example, systemic lupus erythematosus, Sjögren's syndrome, psoriasis, chronic rheumatoid arthritis, multiple sclerosis and the like), inflammatory diseases, shocks (septic shock and the like), and type I IFN-related diseases such as type I diabetes.

The screening method of the present invention is useful in developing a type I IFN production inhibitor based on the novel mechanism of suppression of Spi-B.

The type I IFN production inhibitor of the present invention has been developed on the basis of the mechanism behind induction of type I IFN production in pDC, which reflects a synergistic effect of Spi-B and IRF-7, and is useful as a pharmaceutical such as an antitumor agent and as a test tool for analyzing the mechanism behind type I IFN production.

This application is based on a patent application No. 2008-220193 (filing date: Aug. 28, 2008) filed in Japan, the contents of which are incorporated in full herein.

1. (canceled)

2. A prophylactic/therapeutic agent for a disease associated with excess production of type I IFN, comprising an antisense nucleic acid or siRNA against Spi-B, or an expression vector capable of expressing the same.

3. A method of screening for a substance capable of inhibiting type I IFN production, comprising evaluating whether a test substance suppresses the expression or function of Spi-B, and selecting a substance that suppresses the expression or function of Spi-B as a substance capable of inhibiting type I IFN production.

4. A type I IFN production inducer comprising an expression vector capable of expressing Spi-B and an expression vector capable of expressing IRF-7 in combination.

5. (canceled)

6. An antisense nucleic acid or siRNA against Spi-B, or an expression vector capable of expressing the same, to be used to prevent or treat a disease associated with excess production of type I IFN.

7. (canceled)

8. A method of inhibiting type I IFN production in a mammal, comprising administering to the mammal an effective amount of an antisense nucleic acid or siRNA against Spi-B, or an expression vector capable of expressing the same.

9. A method of preventing or treating a disease associated with excess production of type I IFN in a mammal, comprising administering to the mammal an effective amount of an antisense nucleic acid or siRNA against Spi-B, or an expression vector capable of expressing the same.

10. (canceled)
